(19) United States
(12) Patent Application Publication
(10) Pub. No.: US 2005/0049242 A1
(43) Pub. Date: Mar. 3, 2005

(54) Novel HIV integrase inhibitors and HIV therapy based on drug combinations including integrase inhibitors

(76) Inventors: W. Edward Robinson JR., Irvine, CA (US); Peter J. King, Atlanta, GA (US); Manfred G. Reinecke, Fort Worth, TX (US)

Correspondence Address: STOUT, UXA, BUYAN & MULLINS, LLP, Suite # 310, 4 Venture, Irvine, CA, 92618, US

(21) Appl. No.: 10/672,724

(22) Filed: Sep. 25, 2003

Related U.S. Application Data
(63) Continuation of application No. 09/647,270, filed on Dec. 21, 2000, now abandoned.

(51) Int. Cl. A61K 31/551; A61K 31/522

(52) U.S. Cl. 514/220; 514/263.32

(57) ABSTRACT

The present invention includes a group of novel compounds that are demonstrated to potently and selectively inhibit HIV integrase (IN) activity in vitro and to potently inhibit HIV replication in live, cultured cells at non-toxic concentrations. The novel compounds disclosed include 2,3-di(3,4-dihydroxy-dihydroxydihydrocinnamoyl)-L-tartaric acid, 2,3-di-(3,4-dihydroxybenzoyl)-L-tartaric acid, 2,3-di-(3,4-dihydroxyphenylacetyl)-L-tartaric acid, 2,3-di-(3,4,5-trihydroxybenzoyl-L-tartaric acid, 2,3-dicaffeoyldiamidopropionic acid, 1,2,-dicaffeoyl-L-glyceric acid, bis,-3,4-dicaffeoyldiamidobenzoic acid, di-3,4-dihydroxybenzylidene succinic acid, di-3,4-dihydrodihydroxybenzylidine succinic acid, 2,3-dicaffeoyl-L-serine, bis-dicaffeoyl-L-isoserine and 1,4-dicaffeoyl-L-lysine. Tests of integrase inhibitors with 2′,3′-dideoxycytidine, zidovudine and nelfinavir (protease inhibitor) indicated a potent synergy against reverse transcriptase inhibitor resistant virus. The potential benefit from the addition of integrase inhibitors to combination drug therapies is significant.

### RELATED APPLICATIONS

The present application is a Continuation-In-Part of U.S. Provisional Patent Applications Ser. No. 60/079,764, filed 27 Mar. 1998, and 60/093,208, filed 17 Jul. 1998; these applications are incorporated herein by reference, and priority is claimed from these applications.

This work was supported in part by the Public Health Service, grant AI-41360 from the National Institutes of Allergy and Infectious Diseases, and the Government may have certain rights in this invention.

### BACKGROUND OF INVENTION

1. Field of the Invention

The present invention concerns the medical area of treatment of viral infections and more particularly includes novel inhibitors of HIV integrase and combination drug therapies including integrase inhibitors.

2. Description of the Prior Art

Infection of an individual with the human immunodeficiency virus (HIV) is understood to be the first step toward the development of acquired immunodeficiency ^{syndrome (AIDS) disease. In nearly all cases where individuals receive no treatment for HIV infection, the proliferation of the virus gives rise to AIDS. As of early} 1999, an estimated 33.4 million people are infected with HIV worldwide. Furthermore, the number of people worldwide that became infected with HIV during 1998 is estimated to be 5.8 million. It has also been observed that the annual rate of new infection with HIV in the entire human population is not declining. Despite this fact, the rate of death due to AIDS has begun to drop in some nations, including the United States, primarily through the recent use of combination drug therapies against HIV infection.

The means by which such therapies counter HIV infection is best understood with reference to the biological mechanisms of the HIV life cycle. HIV is a member of a class of infectious agents known as retroviruses. The infectious form of HIV, a virion, is a particle that consists of a viral genome composed of RNA that is surrounded by proteins encoded by the genome. Infection occurs when an HIV virion enters a susceptible host cell, such as a T lymphocyte within the bloodstream. At this point, one of the viral proteins that comprised the virion, reverse transcriptase (RT), synthesizes a double-stranded DNA copy of the HIV RNA genome. The resulting HIV DNA enters the cell nucleus as part of a stable complex with other virion proteins. This complex contains all the necessary molecular apparatus, for integration of the HIV DNA into the host cell's nuclear DNA. Integration, wherein the HIV DNA is covalently inserted into the host cell's genomic DNA, is absolutely required for productive HIV infection. It is only after integration that the HIV DNA can serve as the template for the production of HIV proteins and RNA that will comprise progeny virions. Among these viral proteins is the HIV protease, the activity of which is, necessary for proper formation of new virions. This process, from viral entry to new virion production, is termed viral replication. Upon release from an infected host cell, the newly produced virions are capable of infecting more, previously uninfected host cells. It is through successive rounds of HIV replication and productive host cell infection that HIV disease spreads throughout numerous host cells and ultimately progresses to AIDS disease.

Currently, the best clinical results in HIV treatment come from the use of combination drug therapies. These therapies consist of the simultaneous administration of multiple drugs, which potently and selectively target different elements of the HIV life cycle to disrupt or forestall productive HIV infection and progression to AIDS. The potency of a drug refers to its capacity to act efficaciously at as low a dose as possible, preferably at levels well below those that result in significant amounts of cell death or other signs of cellular toxicity. Selectivity describes the propensity of the drug to act upon a specific target such as unique kind of viral protein. Both of these attributes are of practical importance in the design of therapeutic regimens as they determine the dose, and thus the frequency, cost, and degree of side effects that may occur with use. A further benefit that stems from the use of combinations of drugs derives from a phenomenon known as synergism. With regards to drug therapies, synergism refers to greater than additive effects that are observed when drugs are taken together as opposed to separately. In fact, for HIV, combinations of protease inhibitors and RT inhibitors have demonstrated additive to synergistic effects in vitro, which were similar to their in vivo effects. A practical consequence of such synergism is that in some cases the dosages of the drugs used in combination may be reduced while maintaining a desired level of therapeutic effect, thus reducing cost, dosage frequency, or the occurrence of undesirable side-effects, or all of these.

As an example, one the most potent combinations in current clinical use consists of two different RT inhibitors in conjunction with an HIV protease inhibitor. In this case, the RT inhibitors, selectively target the activity of HIV reverse transcriptase and serve to reduce the rate at which HIV DNA can be produced from HIV RNA, an event that occurs in the early stages of infection within a given host cell. Meanwhile, the protease inhibitor selectively acts on the HIV protease to interfere with the production of viable virions in the latter stages of infection within a given cell. The use of such combination therapies has become more prevalent, and has been facilitated by the increasing variety of antiretroviral drugs that have been approved for use in humans. There are now at least five nucleoside analogue and non-nucleoside analogue HIV RT inhibitors as well as four HIV protease inhibitors approved for use by the US Food and Drug Administration (FDA). Several more HIV protease inhibitors are likely to be approved within the next year or so.

Although the use of combination drug therapies against HIV has proven to be effective in many patients, the current drug regimens are far from ideal. Adherence to these combination regimes is remarkably difficult in terms of patient compliance, and the drug combinations are quite expensive. Their use has been further hampered because many HIV infected individuals have been on single drug therapies in the past and are currently infected with HIV viruses that are resistant to one or more inhibitors, thereby greatly reducing the effectiveness of the combination drug therapy. As with the RT inhibitors, resistance to protease inhibitors can also occur. Beyond these issues, a number of undesirable side effects, including lipodystrophy syndromes, have surfaced as a consequence of long-term use of protease inhibitors at current dosage levels. These issues make therapy with existing antiviral agents complicated at best and impossible at worst.

Access to a larger variety of antiviral agents mitigates these difficulties to some degree, providing new alternative combinations when a given regimen proves ineffective; furthermore, access to a broader spectrum of selectivity in antiviral agents would greatly increase the number of possible therapeutic combinations. Such a broadened spectrum of selectivity could be achieved if HIV proteins other than RT and the viral protease were the targets of therapeutic drugs. The potential for synergism between drugs with three or more targets should be even greater than is currently found with dual target combinations. Such synergism could lead to greater reductions in dosages with a concomitant reduction in cost and potentially the degree of undesirable side effects. Because one of the major limiting factors in anti-HIV combination therapy is the cost of the antiviral agents, such a dosage reduction could substantially increase the number of patients who could affordably be treated with combination therapy regimens. Further, increasing the number of viral targets would decrease the likelihood that viral strains could emerge that are simultaneously resistant to all therapeutic agents.

Although most clinicians and scientists recognize the need for anti-HIV agents targeted at other HIV proteins to add to current combination therapies, no drugs have been approved that target the process of HIV integration. As already noted, this stage of the infectious life cycle is absolutely required for all successive stages and the productive infection within an individual that gives rise to disease progression. In principle, if this stage could be targeted with 100% efficiency, further infection of new host cells and disease progression in general could be eliminated. One HIV protein, the enzyme known as integrase (IN), has been shown to have the minimal activities necessary for integration. In vitro, the enzyme processes HIV DNA for insertion into double stranded DNA that is found within a host cell's nucleus. Further, IN cleaves double stranded DNA and facilitates the insertion of the pre-processed HIV DNA into the cleavage site. Finally, IN covalently links the HIV DNA to both cleaved ends of the host DNA. The remaining step in integration, filling of small gaps in the DNA sequences that flank the inserted HIV DNA, is probably carried out by host cell DNA repair mechanisms.

Candidate therapeutic compounds that selectively target the activities of IN have been widely sought. The major classes of IN inhibitors that have been reported to date include DNA-binding agents, topoisomerase inhibitors, aurintricarboxylic acid and cosalene analogues, caffeic acid phenylethyl ester (CAPE), curcumin, suramin, anthraquinones, and bis-catechols and other hydroxylated aromatic compounds. There have been two chief problems with nearly all of these compounds, both of which stem, in part, from a widespread reliance on ill vitro assays of IN activity that utilize purified IN protein. Foremost, selectivity for the IN protein has been difficult to establish. Even zidovudine and other nucleoside analogues, compounds generally used to target HIV RT, have been reported to inhibit HIV IN in vitro at sufficiently high concentrations. Aurintricarboxylic acid and related compounds also inhibit RT and other phosphoryltransferase enzymes. Inhibition of IN by DNA-binding agents and topoisomerase inhibitors, such as doxorubicin, is relatively weak and nonselective. In virtually all cases, a protective effect of these small molecule IN inhibitors against HIV infection in live, cultured cells has either been undetectable or not examined.

There are a few exceptional compounds that have been demonstrated to inhibit IN activity potently in vitro and HIV replication in live, cultured cells. Octet oligonucleotide inhibitors of HIV IN have been reported that show both kinds of activity and have been entered into phase I clinical trials. Problems attendant with these compounds include high synthetic costs, low bioavailability, and uncertainty about their mechanism of inhibition. In fact, most evidence supports a mechanism of action prior to integration at the level of viral entry. Another class of compounds that holds promise includes the dicaffeoylquinic acids (DCQAs) and the dicaffeoyltartaric acids (DCTAs). These compounds are similar to bis-catechols except that they are more potent, selective inhibitors of HIV IN catalytic activities in vitro and are active in blocking replication of HIV in live, cultured cells at non-toxic concentrations. The compounds disclosed below are all structurally similar to the DCQAs and DCTAs and were developed from studies in which the DCTA L-chicoric acid served as a lead compound.

It is clear that new compounds that have potent anti-HIV activity, both i) vitro and in live cell studies, and that are selective for inhibition of IN would be valuable as anti-HIV therapeutics or lead compounds for anti-HIV therapeutic development. In cases where such pharmacologic inhibitors of IN show demonstrable synergism with existing drugs (or those currently in development) that target other aspects of the HIV life cycle, including the HIV RT inhibitors and HIV protease inhibitors, significant reductions in cost could result in a wider availability of these treatment regimens among a larger number of patients. Even in the absence of synergism with existing anti-HIV agents, the power of the existing drugs makes it unlikely that any new anti-HIV agent will be introduced into patients unless that agent works in combination with existing anti-HIV therapeutics. Also disclosed herein are data demonstrating that the lead DCTA compound L-chicoric acid can act synergistically in combination with established RT inhibitors, protease inhibitors, and simultaneously with both kinds of these established anti-HIV therapeutics. It is expected that the novel compounds presented herein will show behavior similar to L-chicoric acid. Further it is expected that these compounds will serve as leads for the development of a greater variety of potent, selective inhibitors of HIV IN.

### BRIEF SUMMARY OF INVENTION

The present invention includes a group of novel compounds that are demonstrated to potently and selectively inhibit HIV integrase (IN) activity in vitro and to potently inhibit HIV replication in live, cultured cells at non-toxic concentrations. These novel, biologically active IN inhibitors are structural analogues of the selective and potent anti-HIV IN compound, L-chicoric acid. Additionally, the use of IN inhibitors of this class in combination with established anti-HIV therapeutics to form synergistic combinations that inhibit HIV replication in live, cultured cells is demonstrated for the first time. The use of this novel class of IN inhibitors in combination with the established classes of anti-HIV compounds, namely reverse transcriptase (RT) inhibitors and protease inhibitors, to interfere with HIV replication provides a synergistic combination therapy.

In the course of the studies leading up to the present invention, a large number of different analogues of L-chichoric acid were synthesized and investigated in terms of in vitro ability to inhibit IN and ability within cell culture to decrease cell death caused by HIV. A number of novel compounds were found that show favorable properties in the assays. Because of their different structural properites some are expected to show superior properties in actual therapeutic regimes. Particularly interesting novel compounds include 2,3-di(3,4-dihydroxydihydroxydihydrocinnamoyl)-L-tartaric acid, 2,3-di-(3,4-dihydroxybenzoyl)-L-tartaric acid, 2,3-di-(3,4-dihydroxyphenylacetyl)-L-tartaric acid, 2,3-di-(3,4,5-trihydroxybenzoyl-L-tartaric acid, 2,3-dicaffeoyldiamidopropionic acid, 1,2,-dicaffeoyl-L-glyceric acid, bis,-3,4-dicaffeoyldiamidobenzoic acid, di-3,4-dihydroxybenzylidene succinic acid, di-3,4-dihydrodihydroxybenzylidine succinic acid, 2,3-dicaffeoyl-L-serine, bis-dicaffeoyl-L-isoserine and 1,4-dicaffeoyl-L-lysine.

An especially exciting discovery concerns the combination of the integrase inhibitors with other HIV therapeutics such as reverse transcriptase inhibitors and protease inhibitors. Tests of integrase inhibitors with 2′,3′-dideoxycytidine, zidovudine and nelfinavir (protease inhibitor) indicated a potent synergy against reverse transcriptase inhibitor resistant virus. The potential benefit from the addition of integrase inhibitors to combination drug therapies is significant.

### BRIEF DESCRIPTION OF THE DRAWINGS

FIG. 1a shows a structural diagram of compounds number 1, 2, and 3.

FIG. 1b shows a structural diagram of compound number 19.

FIG. 1c shows a structural diagram of compound number 22.

FIG. 2a shows a structural diagram of compound number 25.

FIG. 2b shows a structural diagram of compound number 26.

FIG. 2c shows a structural diagram of compound number 28.

FIG. 3a shows a structural diagram of compound number 35.

FIG. 3b shows a structural diagram of compound number 36.

FIG. 3c shows a structural diagram of compound number 37.

FIG. 4a shows a structural diagram of compound number 38.

FIG. 4b shows a structural diagram of compound number 39.

FIG. 4c shows a structural diagram of compound number 40.

FIG. 4d shows a structural diagram of compound number 41.

FIG. 5 is a histogram showing the anti-HIV activity of several established anti-HIV therapeutics and L-CCA against 4 different HIV molecular clones.

FIG. 6 is a graph of the anti-HIV activity, as measured by RT release, of various drug combination regimens against HIV_{LAI}. Each point is the mean of triplicate infections; error bars indicate standard deviations.

FIG. 7 is a graph of the anti-HIV activity, as measured by RT release, of various drug combination regimens against HIV_{LAI}. Each point is the mean of triplicate infections; error bars indicate standard deviations.

FIG. 8 is a graph of the anti-HIV activity, as measured by RT release, of various drug combination regimens against the clinically isolated strain HIV_{R45}. Each point is the mean of triplicate infections; error bars indicate standard deviations.

FIG. 9 is a graph of the anti-HIV activity, as measured by RT release, of various drug combination regimens against the clinically isolated strain HIV_{R19}. Each point is the mean of triplicate infections; error bars indicate standard deviations.

FIG. 10 is a diagram of Scheme 1 showing the steps in the chemical synthesis of some of the compounds disclosed in the present invention. Abbreviations of compounds are as follows: OM=O-methoxycarbonyl=—OCOOMe; DPM=‘diphenylmethyl’=—CHPh₂; CAF=caffeoyl=—COCH═CH-[3,4-(OH)₂-phenyl]; BMC=bismethoxycarbonylcaffeoyl=—COCH═CH-[3,4-(OM)₂-phenyl]. Components of procedures are abbreviated: [A]: CICOOMe; [B]: Na₂CO₃; [C]: Ph₂CHN₂; [D]: HOAc; [E]: RCOCl; [F]: RCOCl/R₃N; [G]: L-tartaric acid; [H]: [COCl]₂; [I]: SOCl₂.

FIG. 11 is a diagram of Scheme 2 showing the steps in the chemical synthesis of some of the compounds disclosed in the present invention. Abbreviations of compounds are as follows: OM=O-methoxycarbonyl=—OCOOMe; DPM=diphenylmethyl=—CHPh₂; CAF=caffeoyl=—COCH═CH-[3,4-(OH)₂-phenyl]; BMC=bismethoxycarbonylcaffeoyl=—COCH═CH-[3,4-(OM)₂-phenyl]. Components of procedures are abbreviated: [A]: CICOOMe; [B]: Na₂CO₃; [C]: Ph₂CHN₂; [D]: HOAc; [E]: RCOCl; [F]: RCOCl/R₃N; [G]: L-tartaric acid; [H]: [COCl]₂; [I]: SOCl₂.

FIG. 12 is a diagram of Scheme 3 showing the steps in the chemical synthesis of some of the compounds disclosed in the present invention. Abbreviations of compounds are as follows: OM=O-methoxycarbonyl=—OCOOMe; DPM=diphenylmethyl=—CHPh₂; CAF=caffeoyl=—COCH═CH-[3,4-(OH)₂-phenyl]; BMC=bismethoxycarbonylcaffeoyl=—COCH═CH-[3,4-(OM)₂-phenyl]. Components of procedures are abbreviated: [A]: CICOOMe; [B]: Na₂CO₃; [C]: Ph₂CHN₂; [D]: HOAc; [E]: RCOCl; [F]: RCOCl/R₃N; [G]: L-tartaric acid; [H]: [COCl]₂; [I]: SOCl₂.

FIG. 13 is a diagram of Scheme 4 showing the steps in the chemical synthesis of some of the compounds disclosed in the present invention. Abbreviations of compounds are as follows: OM=O-methoxycarbonyl —OCOOMe; DPM=diphenylmethyl=—CHPh₂; CAF=caffeoyl=—COCH═CH-[3,4-(OH)₂-phenyl]; BMC=bismethoxycarbonylcaffeoyl=—COCH═CH-[3,4-(OM)₂-phenyl]. Components of procedures are abbreviated: [A]: ClCOOMe; [B]: Na₂CO₃; [C]: Ph₂CHN₂; [D]: HOAc; [E]: RCOCl; [F]: RCOCl/R₃N; [G]: L-tartaric acid; [H]: [COCl]₂; [I]: SOCl₂.

FIG. 14 is a graph of HIV_{NL4-3} passaged in the presence (squares) or absence (circles) of increasing concentrations of L-chicoric acid. Each point is the mean of triplicate samples; bars are one standard deviation.

FIG. 15 is a schematic diagram of the cloning strategy used to analyze mutations in HIV IN.

FIG. 16a is a graph showing the relative resistance of 4 molecular clones of HIV to the RT inhibitor zidovudine.

FIG. 16b is a graph showing the relative resistance of 4 molecular clones of HIV to the IN inhibitor L-chicoric acid.

The invention and its various embodiments may now be understood by turning to the following Detailed Description of the Preferred Embodiments.

### DETAILED DESCRIPTION OF THE PREFERRED EMBODIMENTS

The present invention generally related to integrase inhibitors having the general structural formula (formula(I)): 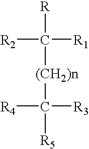
Wherein,
R₁ and R₃ are selected from hydrogen, OR₆, NR₆ and aralkyl groups, optionally substituted with between one and three substituents selected from hydroxyl, halo, lower alkoxy, lower alkylcarbonyloxy and lower alkoxycarbonyloxy groups;
R and R₅ are selected from hydrogen, COOR₇ and CONHR₇;
R₂ and R₄ are hydrogen or may combine with each other to form a cycloalkyl ring or with R₁ and R₄₁ respectively, to form aromatic rings optionally substituted with from one to three substituents selected from OR₆ and NR₆ groups;
R₆ is selected from 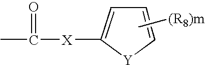
   where,
X is a saturated or unsaturated, acyclic or cyclic, straight or branched, chiral or achiral hydrocarbyl group with from 0 to 10 carbon atoms, and
Y is selected from CH═CH, n=CH, CH═N, O, S, or NR₇. n is between 0 and 4, and m is between 0 and 3.
R₇ is selected from hydrogen, alkyl and aralkyl groups;
R₈ is selected from hydrogen, hydroxy, halo, lower alkoxy, lower alkycarbonyloxy and lower alkoxycarbonyloxy or a cyclic carbonate group when the hydroxy groups are on adjacent carbons;

Additionally, when R and R₅ are COOR₇ or CONHR₇, the groups R₁, R₂ and R₃, R₄ may combine to form an arylidene group, optionally substituted from 1 to 3 substituents selected from hydroxy, halo, lower alkoxy, lower alkycarbonyloxy and lower alkoxycarbonyloxy.

A preferred group of compounds of formula (I) is that wherein, R and R₅ are COOR₇ and CONR₇RG; R₂ and R₄ are hydrogens; R₁ and R₃ are aralkyl or R₁, R₂ and R₃, R₄ may combine to form an arylidene group, optionally substituted with up to 3 hydroxy, alkylcarbonyloxy or alkoxycarbonyloxy groups or OR₆ or NR₆; R₆ is 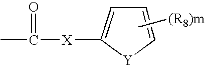
where, X is saturated or unsaturated hydrocarbyl group with from 0 to 4 carbon atoms, Y is CH═CH, CH═N, N═CH, O, S or NR₇; R₇ and R₉ are same or different and may be hydrogen, lower alkyl and alkenyl or aralkyl or R₇ and R₉ may combine to form a heterocyclic ring, optionally substituted with one or more lower alkyl with from 1 to 5 carbon atoms, aralkyl and hydroxy groups; R₈ represents up to 3 hydroxy, alkylcarbonyloxy or alkoxycarbonyloxy groups and n is 0.
Examples Include:
2,3-di-3-(3,4-dihydroxyphenyl)-2-propenoyl L-tartaric acid (Dicaffeoyl-L-tartaric acid)
2,3-di-3-(3,4-dihydroxyphenyl)-2-propenoyl D-tartaric acid (Dicaffeoyl-D-tartaric acid)
2,3-di-3-(3,4-dihydroxyphenyl)-2-propenoyl meso-tartaric acid (Dicaffeoyl-meso-tartaric acid)
2,3-di-3-(3,4-dihydroxyphenyl)propanoyl L-tartaric acid [Bis-2,3-(3,4-dihydroxydihydrocinnamoyl)-L-tartaric acid]
2,3-di-3,4-dihydroxybenzoyl L-tartaric acid [Bis-(3,4-dihydroxybenzoyl) L-tartaric acid]
2,3-di-(3,4-dihydroxyphenyl)acetyl L-tartaric acid [Bis-(3,4-dihydroxyphenylacetyl) L-tartaric acid]
2,3-di-3,4,5-trihydroxybenzoyl L-tartaric acid (Digalloyl-L-tartaric acid)
2,3-di-2,3-dihydroxybenzoyl L-tartaric acid [Bis-(2,3-dihydroxybenzoyl) L-tartaric acid]
2,3-di-3,4-dihydroxybenzylidene succinic acid (di-3,4-dihydroxybenzylidene succinic acid)%
2,3-di-3,4-dihydroxybenzyl succinic acid and esters and pharmaceutically acceptable salts thereof.

Yet another preferred group of compounds of formula (I) is that wherein, R, R₂, R₄ and R₅ are hydrogens or R₂ and R₄ may combine to form a cycloalkyl ring or with R₁ and R₂ may form an aromatic ring substituted with up to 3 OR₆ or NR₆ groups; R₁ and R₃ are OR₆ or NR₆, where R₆, X, Y, R₈ and m are described as earlier and n is 0-4.

Examples Include:

1,2-Dihydroxyethyl 3-(3,4-dihydroxyphenyl)-2-propenoate (1,2-Dicaffeoylethaniediol)
1,3-Dihydroxypropyl 3-(3,4-dihydroxyphenyl)-2-propenoate (1,3-Dicaffeoylpropanediol)
1,4-Dihydroxybutyl 3-(3,4-dihydroxyphenyl)-2-propenoate (1,4-Dicaffeoylbutanediol)
trans-1,4-Dihydroxycyclohexyl 3-(3,4-dihydroxyphenyl)-2-propenoate (1,4-trans-Dicaffeoylcyclohexanediol)
cis-1,4-Dihydroxycyclohexyl 3-(3,4-dihydroxyphenyl)-2-propenoate (1,4-cis-Dicaffeoylcyclohexanediol)
trans-1,3-Dihydroxycyclohexyl 3-(3,4-dihydroxyphenyl)-2-propenoate (1,3-trans-Dicaffeoylcyclohexanediol)
cis-1,3-Dihydroxycyclohexyl 3-(3,4-dihydroxyphenyl)-2-propenoate (1,3-cis-Dicaffeoylcyclohexanediol)
trans-1,2-Dihydroxycyclohexyl 3-(3,4-dihydroxyphenyl)-2-propenoate (1,2-trans-Dicaffeoylcyclohexanediol)
cis-1,2-Dihydroxycyclohexyl 3-(3,4-dihydroxyphenyl)-2-propenoate (1,2-cis-Dicaffeoylcyclohexanediol)
1-Carboxyphenyl-3,5-di-3-(3,4-dihydroxyphenyl)-2-propenoate (3,5-Dicaffeoyl benzoic acid)
1-Carboxyphenyl-3,4-di-3-(3,4-dihydroxyphenyl)-2-propenoate (bis-3,4-Dicaffeoyl benzoic acid)
1-Carboxyphenyl-3,4-di-3-(3,4-dihydroxyphenyl)-2-propenamide (bis-3,4-Dicaffeoyl benzoic acid) and esters and pharmaceutically acceptable salts thereof.

Yet another preferred group of compounds of formula (I) is that wherein, R is COOR₇ and CONR₇R₉; R₁, R₄ and R₅ are hydrogens; R₂ and R₃ are OR₆ and NR₆; R₆, X, Y, R₇, R₈, R₉ and m are as defined earlier and n is 0-4.

Examples Include:

1-Carboxy-2-[3-(3,4-dihydroxyphenyl)-2-propenoyl]amino-3-propyl[3-(3,4-dihydroxyphenyl)-2-propenoate (2,3-Dicaffeoyl-L-serine)
1-Carboxy-3-[3-(3,4-dihydroxyphenyl)-2-propenoylamino]-2-propyl [3-(3,4-dihydroxyphenyl)-2-propenoate (bis-Dicaffeoyl-L-isoserine)
2,3-Di-[3-(3,4-dihydroxyphenyl)-2-propenoyl]aminopropanoic acid (2,3-Dicaffeoyldiamidopropionic acid)
1-Carboxypropyl di-2,3-[3-(3,4-dihydroxyphenyl)-2-propenoate] (1,2-Dicaffeoyl-L-glyceric acid)
2,6-Di-[3-(3,4-dihydroxyphenyl)-2-propenoyl]aminohexanoic acid (1,4-Dicaffeoyl-L-lysine)

It will be readily appreciated by those of skill in the art that certain compounds represented by the above formula may exhibit optical and geometric isomerism. However, when no designation of isomers is specified with respect to the compounds of the present invention, it is to be understood that all possible stereoisomers (R, S), geometric isomers (E, Z) and racemates are included within the invention.

In addition, certain of the compounds that fall within the described bounds of the present invention may form salts with organic and inorganic bases, and it is specifically contemplated that all such salts, in particular physiologically acceptable salts, are included within the present invention.

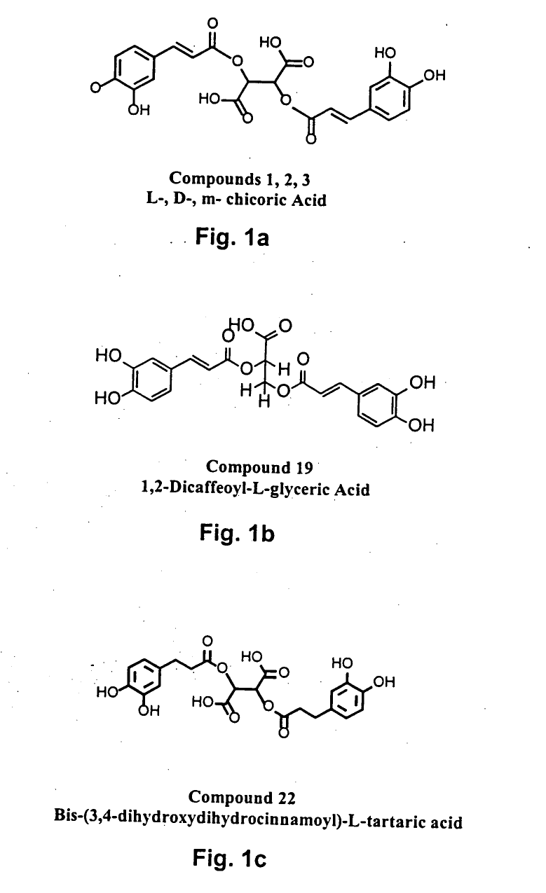

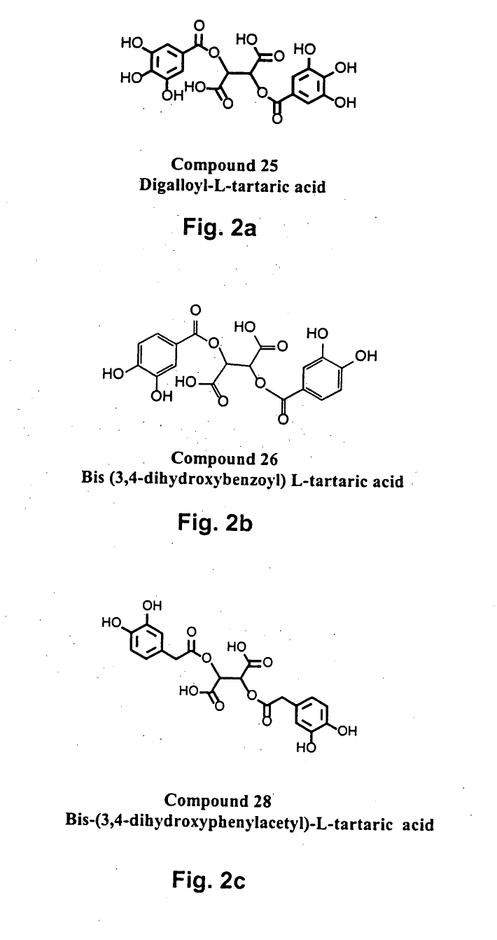

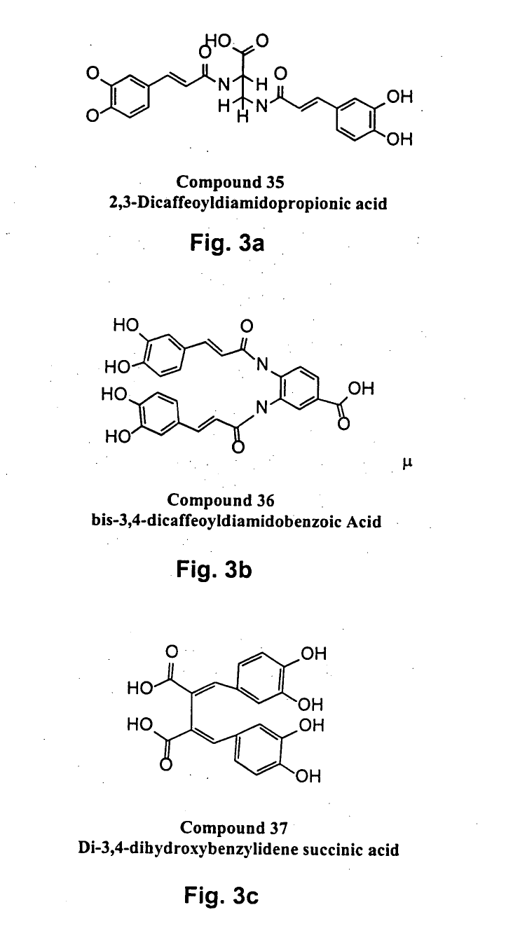

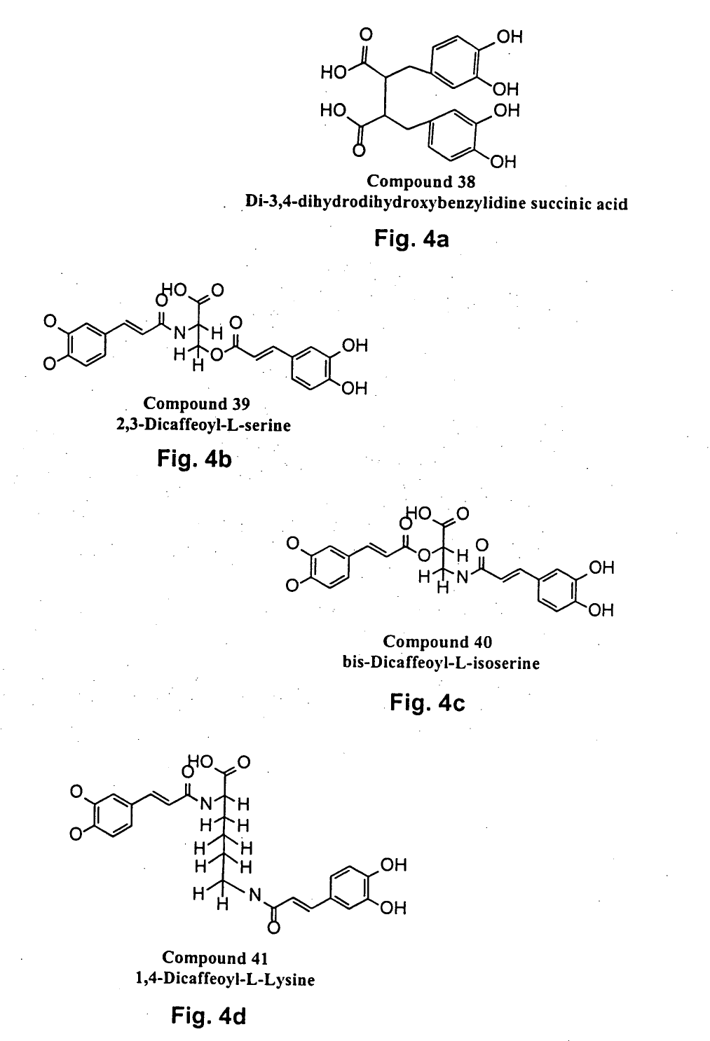

The chemical structures of the compounds are illustrated in FIGS. 1-4. In FIG. 1a, the D- and meso-isomers of L-chicoric acid are identical in this illustrative format. Pharmacological data about the compounds that constitute the invention can be found in Table 1. Table 1 lists the compounds by name, with a chemical identification number keyed to the drawings, LD₅, ED₅₀, and IC₅₀. The first compound in Table 1, L-chicoric acid, a previously described DCTA, is included for reference. LD₅, the 5% lethal dose, is a measure of the toxicity of compound to living, cultured cells. It is the concentration at which cell growth is inhibited by 5%. This represents a truly non-toxic dose as it is within one standard deviation of the growth observed for cells cultured in the absence of the compound. ED₅₀, the 50% effective dose, is the concentration of compound that inhibits HIV-induced cell death by 50% with respect to control HIV infections of cells monitored in the absence of compound. IC₅₀, the 50% inhibitory dose, is the concentration of compound that is observed to inhibit purified HIV IN activity iin vitro in an assay of IN dependent disintegration. Most simply understood, disintegration is a term used to refer to the reverse of integration.

The biological role of IN is to catalyze the insertion of viral DNA into the double-stranded, nuclear DNA of a host cell, while leaving flanking free viral 5′ ends. However, because IN is a true enzyme, it can also catalyze the reverse reaction, namely, excision of viral-like DNA from a double-stranded DNA substrate where free viral-like 5′ ends occur. The use of an appropriately designed DNA substrate allows for IN dependent disintegration to be measured. Not all of the possible enzymatic functions of IN are completely understood; therefore, it is not warranted to state that in vitro measurement of disintegration inhibition is a complete measure of IN inhibition. Nonetheless, the compounds presented herein generally show a strong correlation between in vitro potency as measured by inhibition of disintegration, and biological potency as measured by inhibition of HIV replication in living, cultured cells. Furthermore, for those compounds in which other measures of IN activity (3′-end processing and strand transfer) have been measured there has been a 1:1 correlation between inhibition of these steps and disintegration.

All of the compounds included in Table 1 inhibit HIV IN dependent disintegration by 90% or more when tested at 25 μM concentration. This is an in vitro measure of potency. The IC₅₀ is also a measure of in vitro potency. This value is significantly smaller than the LD₅ for all the compounds suggesting that each of the compounds may serve to inhibit HIV replication in cells through HIV IN inhibition at non-toxic concentrations. Biological potency can be more directly assessed by reference to the LD, and ED₅₀. In all cases, the ED₅₀ is less than or equal to the LD₅, thus, with these compounds a 50% effective dose is expected to cause little or no significant cell toxicity. In general, the wider the margin of LD₅ over ED₅₀, the greater the concentration of the compound that can be used without danger of causing cell death.

TABLE 1

| | ID | LD₅ | | |
|---|---|---|---|---|
| Compound Name | number | (μM) | ED₅₀ (μM) | IC₅₀ (μM) |
| L-chicoric acid | 1 | 264 | 4.2 | 0.18 |
| D-chicoric acid | 2 | 115 | 6.3 | 0.07 |
| m-chicoric acid | 3 | 373 | 4.2 | 0.08 |
| 1,2-dicaffeoyl-L-glyceric | 19 | 145 | 2.3 | 0.520 |
| acid | | | | |
| Bis-(3,4- | 22 | >263 | 8.4 | 2.38 |
| dihydoxydihydrocinnamoyl)- | | | | |
| L-tartaric acid | | | | |
| Digalloyl-L-tartaric acid | 25 | 2.7 | 0.66 | 0.97 |
| Bis-(3,4-dihydroxybenzoyl)- | 26 | 90 | 0.95 | 0.43 |
| L-tartaric acid | | | | |
| Bis-(3,4- | 28 | 70 | 35 | 0.88 |
| dihydroxyphenylacetyl)-L- | | | | |
| tartaric acid | | | | |
| 2-3- | 35 | 50 | 70 | 0.570 |
| dicaffeoyldiamidopropionic | | | | |
| acid | | | | |
| Bis-3,4- | 36 | 47 | 4.6 | 0.734 |
| dicaffeoyldiamidobenzoic | | | | |
| acid | | | | |
| Di-3,4-dihydroxybenzylidene | 37 | 35 | 8.7 | 2.19 |
| succinic acid | | | | |
| Di-3,4- | 38 | 69 | 4.4 | 0.858 |
| dihydrodihydroxybenzylidine | | | | |
| succinic acid | | | | |
| 2,3-dicaffeoyl-L-serine | 39 | 29 | 2.5 | 0.120 |
| Bis-dicaffeoyl-L-isoserine | 40 | 175 | 3.0 | 0.195 |
| 1,4-dicaffeoyl-L-lysine | 41 | 29.2 | 3.7 | 2.23 |

Many of these compounds appear to selectively inhibit HIV IN as the means by which they inhibit HIV replication in living, cultured cells. This is most strongly indicated for compounds 1, 22, 25, 26, and 28 which all show reduced efficacy against a molecular clone of HIV, HIV_{NL4-3clone1-D4}, that was generated by selection for resistance to L-chicoric acid (compound 1) and was shown to bear a single point mutation in its gene for IN. The single point mutation results in the substitution of a serine amino-acid residue for the normally occurring glycine amino-acid residue at position 140 in the primary sequence of HIV IN. The demonstration that a single amino-acid substitution at this position within IN confers resistance to L-chicoric acid is strong evidence that the principle target for the anti-HIV activity of L-chicoric acid is IN. Likewise, if this mutation confers marked resistance to other anti-HIV compounds the evidence is equally strong that IN is also a target of these compounds. Referring to Table 2 which shows the anti-HIV activity of L-chicoric acid analogues against the L-chicoric acid resistant clone, HIV-1_{NL4-3clone1-D4}, the ED₅₀ for each of the noted compounds is significantly increased when measured against the resistant molecular clone HIV_{NL4-3clone1-D4} as compared to against the parental non-resistant strain HIV_{NL4-3}.

TABLE 2

ED₅₀ (in μM)

| Compound | HIV_{NL4-3}^{a} | HIV_{NL4-3clone1-D4}^{b} | Fold Change |
|---|---|---|---|
| 1 | 0.78 | 61.25 | 78 |
| 22 | 7.8 | 31.25 | 4 |
| 25 | 0.29 | 31.25 | 107 |
| 26 | 0.82 | 52.5 | 64 |
| 28 | 15.6 | 62.5 | 4 |

^{a}The anti-HIV activity of each compound was tested against the infectious molecular clone HIV-1_{NL4-3}. The ED₅₀ is expressed as the mean of triplicate infections.
^{b}The anti-HIV activity of each compound was measured against L-chicoric acid-resistant molecular clone HIV-1_{NL4-3clone1-D4}. The results are expressed as the mean of triplicate infections.

Since combination drug therapies are now the standard of care in the medical treatment of HIV infection, any new therapeutics and therapeutic leads should work in combination with existing classes of anti-HIV drugs. The therapeutic effects of drugs, when used in combination, can generally be described as falling into one of three categories. In the best case, drugs administered in combination can act synergistically, having net therapeutic effects that are greater than the sum of the effects observed when the drugs are administered individually. When drug combinations appear to act with equivalent net therapeutic effects as the sum of the effects when administered individually, the drugs are said to act additively. In the worst case, drugs can act counter-productively, resulting in reduced efficacy when used in combination; this is referred to as antagonism. In general, for the use a drug combination to be warranted, additive to synergistic effects should be demonstrable. Demonstration of such effects in vitro is a first indication that such effects can occur in a medically prescribed therapeutic regimen. As noted, combinations of protease inhibitors and RT inhibitors have previously demonstrated additive to synergistic anti-HIV effects in vitro, which have been similar to their in vivo effects. Similarly, now such anti-HIV activity has been demonstrated for established anti-HIV agents in conjunction with the lead IN inhibitor L-chicoric acid (L-CCA, compound 1). In almost all circumstances tested, L-chicoric acid has acted either synergistically or additively with RT inhibitors and/or protease inhibitors to inhibit HIV replication.

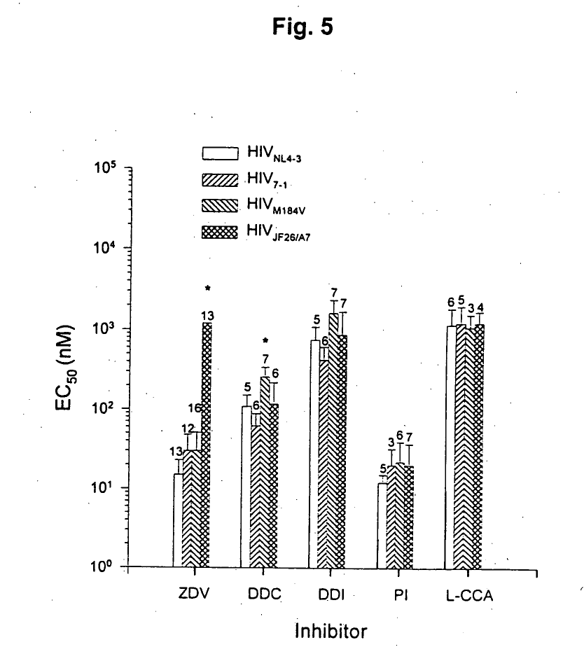

Pairwise combinations of L-CCA with individual RT inhibitors or a protease inhibitor were tested in living cell assays against three different molecular clones of HIV. The molecular clones were HIV_{NL4-3 clone 7-1}, which has silent mutations in the gene for IN and shows an increased susceptibility to the RT inhibitor dideoxycytidine (DDC), HIV_{NL4-3 M185V}, which has a methionine to valine mutation in the gene for RT and shows partial resistance to DDC and another RT inhibitor 2′,3′-dideoxyinosine (DDI), and HIV_{NL4-3 JF26/A7}, which has numerous mutations in the gene for RT and is highly resistant to the RT inhibitor zidovudine (ZDV). For each of these HIV clones, the ED₅₀ was determined for the anti-HIV compounds ZDV, DDC, DDI, the protease inhibitor (PI) Nelfinavir (provided by Agouron Pharmaceuticals, San Diego), and L-CCA. This is shown in FIG. 5. The results of mixed dose effect analyses using the method of Chou and Talalay (Adv. Enzyme Regu. 22:27-55(1984)) are summarized in Table 3 The quantitative data behind this summary is described in the detailed description of the preferred embodiments and in Tables 4-6. Table 4 shows the data for the DDC plus L-chicoric acid combination. Table 5 shows the results for Nelfinavir plus L-chicoric acid. While Table 6 shows the results for Zidovudine plus L-chicoric acid. The principle conclusion from these experiments is that in living cell assays of anti-HIV replication, L-CCA shows synergistic effects when paired with the RT inhibitor DDC and when paired with the protease inhibitor Nelfinavir, against all three molecular clones of HIV. L-CCA also shows synergism when paired with the RT inhibitor ZDV against two of the three HIV clones.

TABLE 3

| Resistance | HIV | ZDV + L-CCA | DDC + L-CCA | L-CCA + PI |
|---|---|---|---|---|
| None | clone7-1 | SI Ant | S | S |
| DDC | M184V | MS | SS | S |
| ZDV | JF26/A7 | S | SS | MS |

DDC = dideoxycytidine
ZDV = zidovudine
L-CCA = L-chicoric acid
PI = protease inhibitor (nelfinavir)
MS = moderate synergism: CI = 0.7-0.85
S = synergy CI = 0.3-0.7
SS = Strong Synergism: CI = 0.1-0.3
VSS = Very Strong Synergism: CI < 0.1
A = additivity: CI = 0.9-1.1
SI Ant = slight antagonism: CI = 1-2.0

TABLE 4

| Virus | Fa* | | DDC | L-CCA |
|---|---|---|---|---|
| (HIV-1) | (protection) | CI^{†} | (nM) | (nM) |
| NL4-3_{clone7-1} | 0.2 | 1.144 (0.35) | 12.4 | 148.3 |
| | 0.4 | 0.726 (0.18) | 21.1 | 253.1 |
| | 0.6 | 0.578 (0.14) | 32.8 | 393.7 |
| | 0.8 | 0.493 (0.13) | 56.0 | 672.0 |
| | 0.9 | 0.456 (0.14) | 87.1 | 1046 |
| NL4-3_{cloneM184V} | 0.2 | 0.576 (0.27) | 21.6 | 64.8 |
| | 0.4 | 0.358 (0.08) | 43.2 | 129.7 |
| | 0.6 | 0.283 (0.05) | 76.8 | 230.3 |
| | 0.8 | 0.249 (0.05) | 153.7 | 461.2 |
| | 0.9 | 0.242 (0.06) | 273 | 819 |
| NL4-3_{cloneJF26/A7} | 0.2 | 2.367 (1.6) | 13.0 | 78.1 |
| | 0.4 | 0.395 (0.1) | 23.8 | 142.8 |
| | 0.6 | 0.214 (0.06) | 39.2 | 235.3 |
| | 0.8 | 0.177 (0.05) | 71.8 | 430.5 |
| | 0.9 | 0.165 (0.09) | 118 | 709 |

*Fa is the fraction affected (i.e. Fa × 100 is the percent protection). Values for 20%, 40%, 60%, 80%, and 90% are indicated.
^{†}Is the combination index. It was calculated using CalcuSyn for Windows and is one representative experiment from a minimum of three. Each experimental value

TABLE 5

| Virus | Fa* | | Nelfinavir | L-CCA |
|---|---|---|---|---|
| (HIV-1) | (protection) | CI^{†} | (nM) | (nM) |
| NL4-3_{clone7-1} | 0.2 | 0.906 (0.29) | 2.9 | 163.6 |
| | 0.4 | 0.472 (0.12) | 4.9 | 279.6 |
| | 0.6 | 0.329 (0.08) | 7.6 | 435.4 |
| | 0.8 | 0.26 (0.08) | 13.1 | 743.9 |
| | 0.9 | 0.239 (0.09) | 20.3 | 1158 |
| NL4-3_{cloneM184V} | 0.2 | 1.81 (0.65) | 2.5 | 144 |
| | 0.4 | 0.91 (0.21) | 4.7 | 269.2 |
| | 0.6 | 0.536 (0.12) | 7.9 | 451.5 |
| | 0.8 | 0.293 (0.08) | 14.8 | 844.1 |
| | 0.9 | 0.183 (0.07) | 24.8 | 1416 |
| NL4-3_{cloneJF26/A7} | 0.2 | 7.982 (5.3) | 4.6 | 264.5 |
| | 0.4 | 1.138 (0.25) | 7.0 | 401.8 |
| | 0.6 | 0.577 (0.13) | 10.0 | 567.8 |
| | 0.8 | 0.444 (0.11) | 15.1 | 862.5 |
| | 0.9 | 0.388 (0.11) | 21.3 | 1219 |
| *Fa is the fraction affected (i.e. Fa × 100 is the percent protection). Values for 20%, 40%, 60%, 80%, and 90% are indicated. | | | | |
| ^{†}Is the combination index. It was calculated using CalcuSyn for Windows and is one representative experiment from a minimum of three. Each experimental value was determined in triplicate. Values in parenthesis are 1.96 × SD as estimated in | | | | |
| # the computer program. | | | | |

TABLE 6

| Virus | Fa* | | Zidovudine | L-CCA |
|---|---|---|---|---|
| (HIV-1) | (protection) | CI^{†} | (nM) | (nM) |
| NL4-3_{clone7-1} | 0.2 | 0.898 (0.37) | 2.7 | 67.2 |
| | 0.4 | 1.023 (0.34) | 8.7 | 217 |
| | 0.6 | 1.197 (0.34) | 22.9 | 571.8 |
| | 0.8 | 1.508 (0.44) | 73.8 | 1845 |
| | 0.9 | 1.865 (0.67) | 194 | 4861 |
| NL4-3_{cloneM184V} | 0.2 | 0.803 (0.39) | 4.9 | 63.2 |
| | 0.4 | 0.508 (0.17) | 10.2 | 132.7 |
| | 0.6 | 0.373 (0.1) | 18.9 | 245.2 |
| | 0.8 | 0.272 (0.07) | 39.6 | 514.8 |
| | 0.9 | 0.217 (0.07) | 73 | 951 |
| NL4-3_{cloneJF26/A7} | 0.2 | 12.6 (8.9) | 150.2 | 450.6 |
| | 0.4 | 1.119 (0.36) | 259.8 | 779.4 |
| | 0.6 | 0.157 (0.08) | 409 | 1226 |
| | 0.8 | 0.017 (0.04) | 707 | 2120 |
| | 0.9 | 0.003 (0.02) | 1112 | 3336 |
| *Fa is the fraction affected (i.e. Fa × 100 is the percent protection). Values for 20%, 40%, 60%, 80%, and 90% are indicated. | | | | |
| ^{†}Is the combination index. It was calculated using CalcuSyn for Windows and is one representative experiment from a minimum of three. Each experimental value was determined in triplicate. Values in parenthesis are 1.96 × SD as estimated in | | | | |
| # the computer program. | | | | |

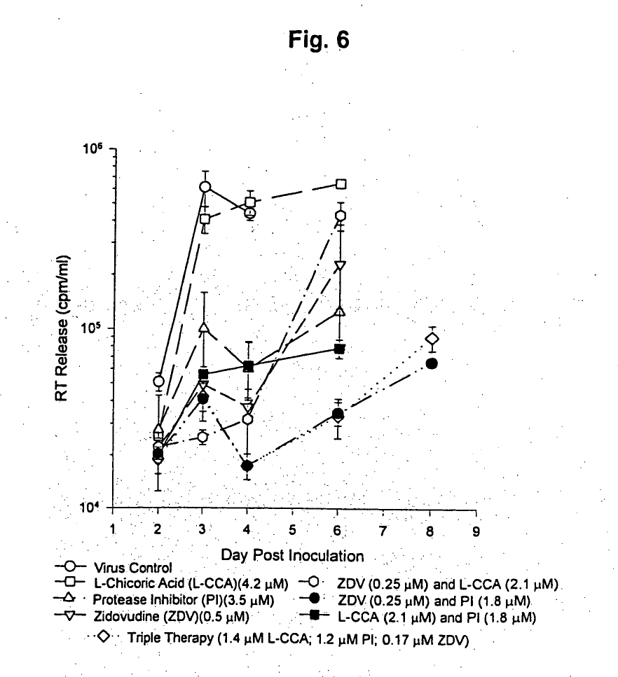

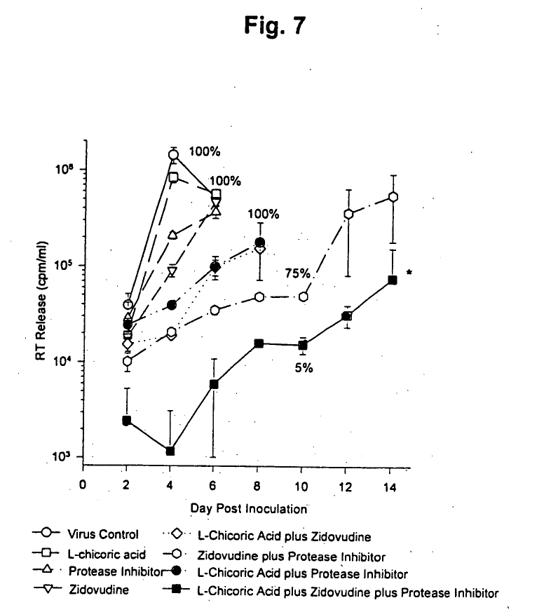

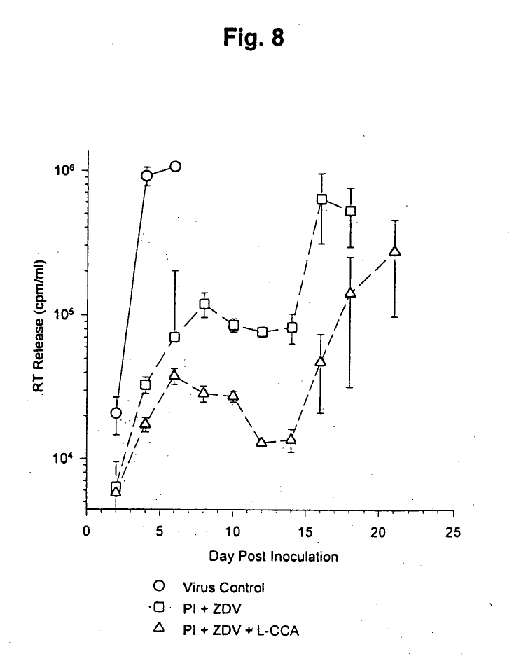

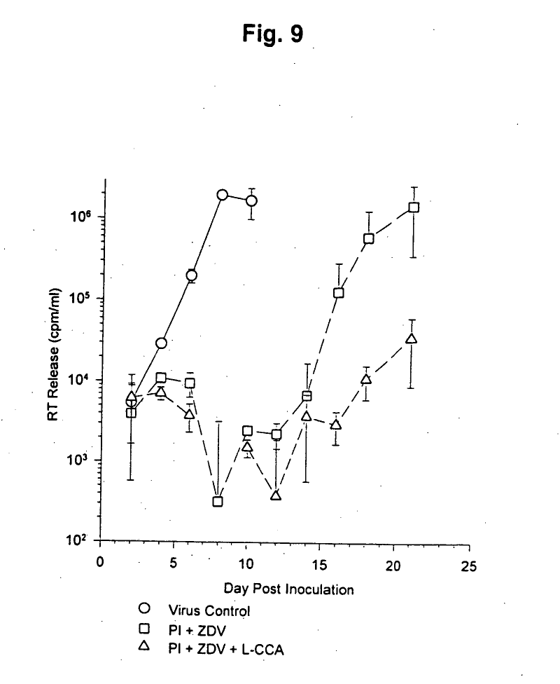

A study has also been conducted that shows synergistic activity of L-CCA against HIV when included in a triple combination of anti-HIV agents. As shown in FIG. 6 against the tissue adapted strain of HIV, HIV_{LAI}, the addition of L-CCA to a combination of zidovdine and the protease inhibitor AG1350 (provided by Agouron Pharamceuticals, San Diego), allowed the dosages of each of the established inhibitors to be reduced by 33% to achieve equivalent anti-HIV replication effects in living, cultured cells. Furthermore, as shown in FIG. 7, when all compounds were administered at constant dose, the triple combination of L-CCA plus ziduvodine plus protease inhibitor showed greater anti-HIV_{LAI} replication potency than any of the compounds individually or in pairwise combination. FIGS. 8 and 9 show that similar results were obtained against two clinically isolated HIV strains, HIV_{R45} and HIV_{R19}. Against both strains, a triple combination including L-CCA, zidovudine and protease inhibitor, had greater anti-HIV replication activity than a dual combination of ZDV and protease inhibitor. The measurements that yielded these results are more completely described in the detailed description of the preferred embodiments.

The lead compound L-CCA has demonstrable compatibility and additive to synergistic effects with compounds from the established classes of anti-HIV agents. The novel, analogue compounds of L-CCA presented generally show equivalent or improved anti-HIV effects consistent with similar potency and selectivity shown when evaluated individually. The translation of such in vitro synergistic effects to in vivo effects has already occurred with combinations of established anti-HIV compounds. The addition of this new class, as represented by the novel compounds introduced here, of IN inhibitors with demonstrable anti-HIV replication activity provides a new avenue for the pursuit and development of clinically valuable anti-HIV therapeutics.

Each novel compound was pursued using the following steps: 1) chemical synthesis, 2) chemical characterization, 3) biological characterization with living cells, including a) determination of LD, by cell toxicity assay, b) determination of ED₅₀ by anti-HIV assay, and c) assessment of selectivity against IN by ED₅₀ determination against the L-chicoric acid (L-CCA) resistant strain, HIV_{NL4-3clone1-D4}, 4) biochemical characterization, i.e., determination of IC₅₀ with the disintegration assay, and 5) determination of pharmacological compatibility of anti-HIV activity when used in combination with members of the established classes of anti-HIV therapeutics, i.e., determination of capacity to act synergistically. This entire course of assessment is not presented for all compounds. Representative examples of these steps and relevant data illustrated by figures and tables are presented below.

Synthesis

In an effort to develop more potent and selective inhibitors of HIV-1 IN, analogues of the DCTA, L-CCA, were synthesized. Exemplary synthetic schemes for a few of these compounds are shown in FIGS. 1-4 and described below. The length of the side chains, spatial arrangement of the phenolic hydroxyl groups, size and structure of the central molecular core structure, and number of free carboxyl groups were all varied. The effects of these changes were assayed against HIV-1 IN in the disintegration reaction as well as against HIV-1 replication and cell growth in tissue culture.

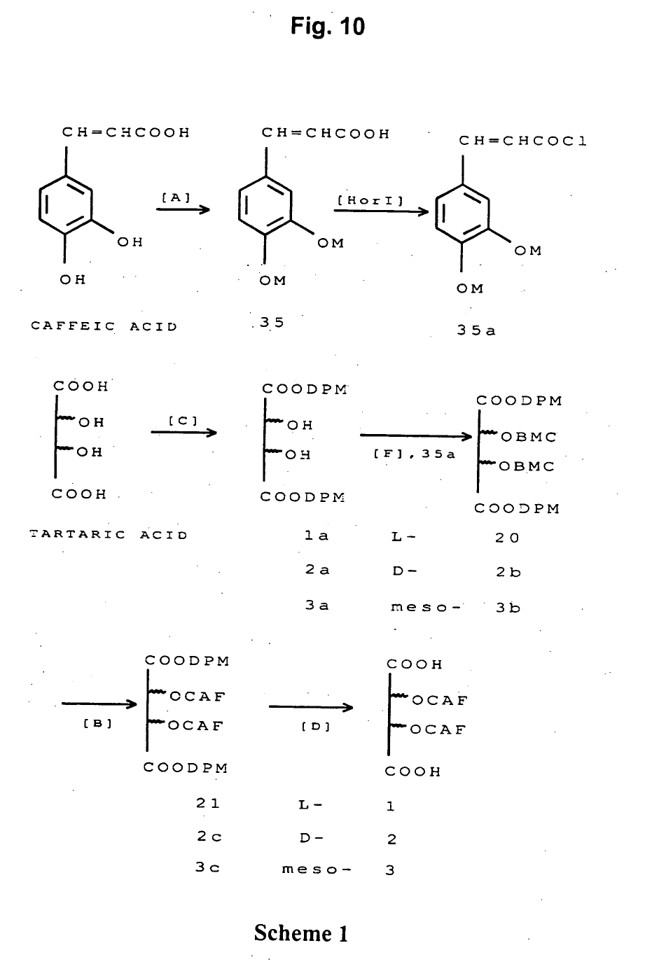

The synthetic schemes which are illustrated in FIGS. 10-13 are briefly described as follows:

L-(1), D-(2)- and meso-(3) DCTA were synthesized by acylation of the bis(diphenylmethyl) tartrates (1a, 2a and 3a) with the protected caffeoyl chloride (35a) to give the fully-blocked compounds (20, 2b and 3b) from which the phenol and carboxyl blocking groups were sequentially removed via (21, 2c and 3c) (FIG. 10, Scheme 1).

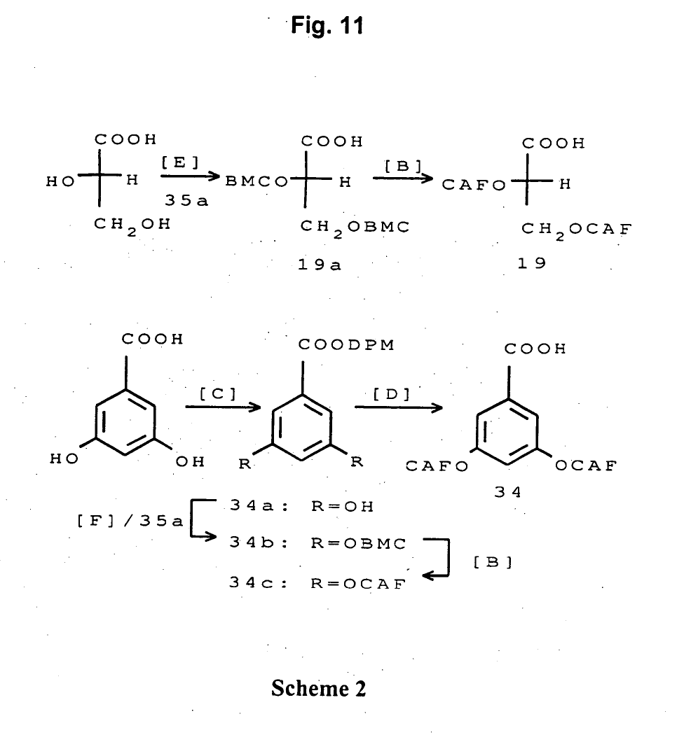

A compound with a single carboxyl group, dicaffeoylglyceric acid (19) was prepared by acylation with (35a) and deprotection via 19a (FIG. 11, Scheme 2).

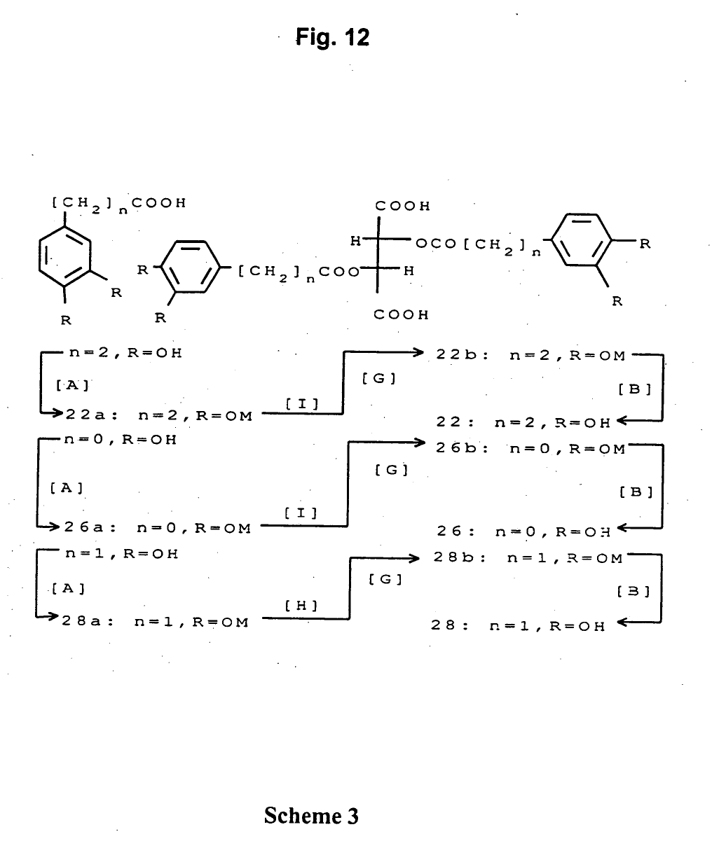

To determine the effect on bioactivity of the group linking the catechol and tartaric acid moieties of 1, analogues were prepared with the CH═CH replaced by two (22), one (28) or zero (26) methylene groups. Direct acylation of L-tartaric acid with the acid chlorides of the phenol protected acids 22a, 28a and 26a gave 22b, 28b and 26b from which the phenol blocking groups were removed (FIG. 12, Scheme 3).

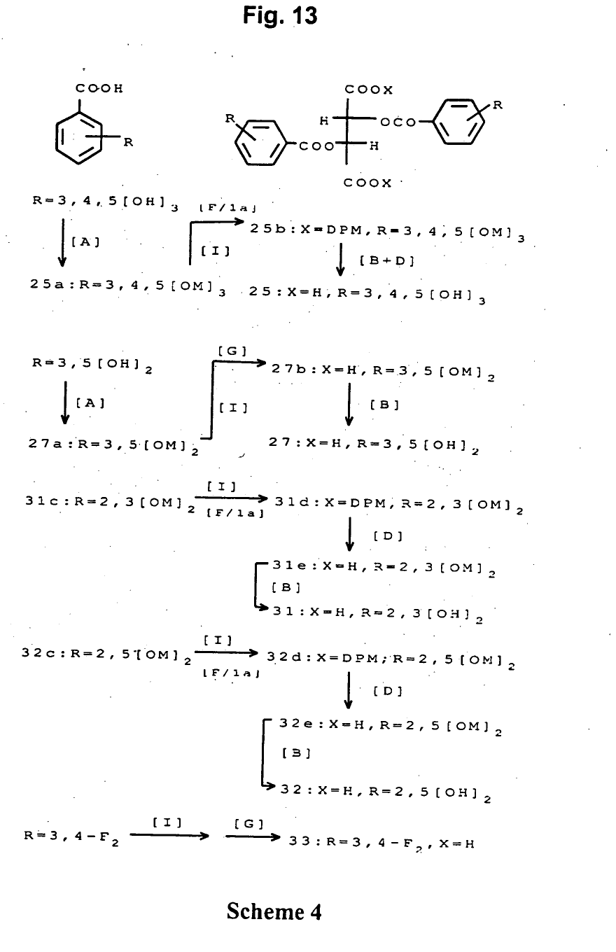

The significance for bioactivity of the number and position of the phenol groups was determined in the benzoic acid series (FIG. 13, Scheme 4). An analogue with three (25) phenolic hydroxyl groups was synthesized by acylation of the diester of L-tartaric acid 1a with the acid chloride of the phenol-protected acid 25a followed by removal of the appropriate blocking groups.

Chemical Procedures:

Detailed synthetic procedures and chemical characterizations, as referred to in FIGS. 10-13, are described as follows.

General. All melting points were measured on a Mel-Temp apparatus and are corrected. Elemental analyses were performed by M-H-W laboratories of Phoenix, Ariz. Mass spectrometry (MS) analyses were obtained on a HP-5989A instrument at 70 eV in the EI mode with GC sampling unless otherwise noted as DIP (direct insertion probe). Selected peaks are reported as m/z (rel.int.) including all (except isotope peaks) with m/z>100 and relative intensity>25. HRFABMS were performed at the Washington University Resource for Biomedical and Bio-Organic Mass Spectrometry, St. Louis, Mo. NMR spectra were obtained on a Varian XL-300 instrument at 299.936 (¹H) or 75.427 (¹³C) unless otherwise noted in the indicated solvent (D=DMSO-d₆, C═CDCl₃, M=CD₃OD) and are reported in order as: ppm downfield from TMS at δ=0, multiplicity (s, d, dd, m, bs), observed couplings J in Hz and relative # of H's. APT spectra results are expressed as d=C or CH₂, u=CH or CH₃. HPLC used a C-18-10 μm, 250 mm×4.6 mm analytical column or a C18-10 μm, 250×22 mm preparative column eluted with either methanol-water or acetonitrile-water mixtures containing 1% acetic acid with UV detection at 254 nm. The following general synthetic procedures refer to both Schemes 1-4 and the description of the preparation of the specific compounds that follows. No attempt was made to optimize yields.

Procedure A, Carboxymethylation of Phenols: A solution of the phenolic acid in 2.2 eq 1N NaOH was cooled to 0° C. and 3.5 eq methyl chloroformate was added drop-wise with stirring. The precipitate was collected by filtration, washed with deionized water, air dried and recrystallized to give the blocked phenolic acid.

Procedure B, Removal of Carboxymethyl Groups: The carboxymethylated phenol was dissolved in tetrahydrofuran (THF) and hydrolyzed with 2% Na₂CO₃ with enough methanol for homogeneity at room temperature, under N₂ for 5-7 hr. The reaction mixture was acidified to pH=1-2 with ION HCl, extracted with ether, the ether evaporated and the residue purified by chromatography on silica gel, if necessary.

Procedure C, Formation of Diphenylmethyl Esters: A solution of 1.25 eq diphenyldiazomethane per COOH group in chloroform was added to the carboxylic acid in MeOH—CHCl₃. The mixture was stirred at room temperature until the red color disappeared, washed successively with 1N HCl, saturated NaHCO₃ and H₂O. The organic layer was dried with Na₂SO₄ and evaporated on a Rotovap to afford the diphenylmethyl ester which was recrystallized if necessary.

Procedure D, Hydrolysis of Diphenylmethyl Esters: The diphenylmethyl esters were deprotected in refluxing 70% acetic acid under N₂ for 4 hours. The solvent was removed by lyophilization and the residue purified by chromatography on Sephadex LH-20.

Procedure E, Direct Acylation of Alcohol Groups: The alcohol was reacted with a slight excess of the acid chloride without solvent in an oil bath at 130-140° C. for 15 min and the residue chroinatographed on silica gel.

Procedure F, Solution Acylation of Alcohol Groups: The acyl chloride was reacted with the alcohol in anhydrous be nzene with pyridine or triethylamine as a catalyst for 2.5 hr at room temperature. The reaction mixture was successively washed with 1N HCl, saturated NaHCO₃ and water and the organic layer evaporated to give the ester which was purified on a silica gel column.

Procedure G, Direct Ac lation of L-Tartaric Acid: A modification of Scarpati's method (Tetrahedron 4:43-48 (1958)) involves heating L-tartaric acid with an excess of an acyl chloride in an oil bath at 135-160° C. for 10-30 min followed by hydrolysis of the intermediate anhydride with 80% HOAc on a steam bath for 30 min. The residue from removal of the solvent below 40° C. under reduced pressure was partitioned between water andether and the latter dried and evaporated to give a crude product which was purified by chromatography on silica gel or Sephadex LH-20.

Procedure H, Formation of Acyl Chlorides with Oxalyl Chloride: A solution of the acid in excess oxalyl chloride was stirred with a 25-fold excess of oxalyl chloride at room temperature for one hour, the excess reagent removed on a Rotovap and the acid chloride used immediately without purification.,

Procedure I, Formation of Acyl Chlorides with Thionyl Chloride: A solution of the acid in excess thionyl chloride was heated (NaOH trap) in an oil bath at 80-90° C. until HCl evolution ceased. Removal of the thionyl chloride on a Rotovap gave the acid chloride used immediately without purification.

Specific Syntheses and Chemical Characterizations:

Dicaffeoyl-L-tartaric acid (L-chicoric acid, L-DTCA or L-CCA) (1). L-Tartaric acid was converted by Procedure C to bis(diphenylmethyl) L-tartrate (1a) as a white powder, mp=107-108° C.; ¹H NMR (C): 7.24-7.32 (m, 20H), 6.98 (s, 2H), 4.75 (s, 2H); ¹³C(C): 170.7, 139.0, 138.9, 128.6, 128.5, 128.4, 128.1, 127.6, 126.9, 79.2, 72.3; DIP/MS: 315 (2), 183 (33), 167 (100), 165 (43). Reaction of 1a with 35a via Procedure F gave bis(diphenylmethyl) bis [di(methoxycarbonyl)caffeoyl]-L-tartrate (20), mp=62-64° C.; ¹H NMR (C): 7.50 (d, 16.0, 2H), 7.10-7.38. (m, 20H), 6.94 (s, 2H), 6.16 (d, 16.0, 2H), 6.04 (s, 2H), 3.93 (s, 6H), 3.92 (s, 6H); ¹³C(C): 164.8 (×2), 153.1, 152.9, 144.3, 143.9, 142.7, 138.8, 138.7, 133.0, 128.63, 128.57, 128.2 (×2), 127.3, 127.0 (×2), 123.50, 122.6, 117.5, 79.1, 71.1, 56.0 (×2). Removal of the methoxycarbonyl groups by Procedure B gave bis(diphenylmethyl) dicaffeoyl-L-tartrate (21), mp=135-136° C. (dec.), ¹H NMR (M): 7.47 (d, 15.8, 2H), 6.91 (s, 2H), 6.79-7.36 (m, 20H), 6.10 (s, 2H), 6.07 (d, 15.8, 2H); ¹³C (M): 167.4d, 166.8d, 150.2d, 148.8u, 146.9d, 140.7d (×2), 129;7u (×2), 129.2u (×2), 128.2u, 128.0u, 127.5d, 123.6u, 116.6u, 115.4u, 113.3u, 80.3u, 72.5u. Removal of the diphenylmethyl groups by Procedure D gave L-DCTA (1), whose ¹H and ¹³C NMR agreed with the literature.

Dicaffeoyl-D-tartaric acid (D-chicoric acid, D-DCTA) (2). D-Tartaric acid was converted by Procedure C to bis(diphenylmethyl) D-tartrate (2a) as a white powder, mp=108-110° C.; ¹H NMR (C): 7.28-7.34 (m, 20H), 6.99 (s, 2H), 4.76 (s, 2H); ¹³C(C): 170.7, 139.0, 138.9, 128.6, 128.5, 128.4, 128.1, 127.6, 126.9, 79.2, 72.3; DIP/MS: 315 (2), 183 (37), 167 (100), 165 (47). Reaction of 2a with 35a via Procedure F gave bis(diphenylmethyl) bis[di(methoxycarbonyl)caffeoyl]-D-tartrate (2b) mp=61-63° C.; ¹H NMR (C): 7.50 (d, 16.0, 2H), 7.09-7.37 (m, 20H), 6.94 (s, 2H), 6.16 (d, 16.0, 2H), 6.04 (s, 2H), 3.93 (s, 6H), 3.92 (s, 6H); ¹³C(C): 164.8 (×2), 153.1, 152.9, 144.3, 143.9, 142.7, 138.8, 138.7, 133.0, 128.63, 128.57, 128.2 (×2), 127.3, 127.0 (×2), 123.50, 122.6, 117.5, 79.1, 71.1, 56.0 (×2). Removal of the methoxycarbonyl groups by Procedure B gave bis(diphenylmethyl) dicaffeoyl-D-tartrate (2c), mp=134-136° C. (dec.); ¹H NMR (M): 7.49 (d, 15.8, 2H), 6.90 (s, 2H), 6.80-7.33 (m, 20H), 6.08 (s, 2H), 6.07 (d, 15.8, 2H); ¹³C (M): 167.4d, 166.8d, 150.0d, 148.7u, 146.8d, 140.6d, 140.5d, 129.6u (×2), 129.1u (×2), 128.1u, 127.9u, 127.4d, 123.6u, 116.5u, 115.3u, 113.2u, 80.4u, 72.5u. Removal of the diphenylmethyl groups by Procedure D gave D-DCTA (2), whose ¹H and ¹³C NMR agreed with the literature.

Dicaffeoyl-meso-tartaric acid (meso-chicoric acid, meso-DCTA) (3). meso-Tartaric acid was converted by Procedure C to bis(diphenylmethyl) meso-tartrate (3a) as a white powder, mp=108-109° C.; ¹H NMR (C): 7.18-7.27 (m, 20H), 6.82 (s, 2H), 4.72 (s,2H); ¹³C(C): 170.2, 139.0, 138.7, 128.52, 128.45, 128.23, 128.18, 128.0, 127.2, 79.4, 73.1; DIP/MS: 315 (2), 183 (37), 167 (100), 165 (47); DIP/MS: 315 (2), 183 (35), 167 (100), 165 (48). Reaction of 3a with 35a via Procedure F gave bis(diphenylmethyl) bis[di(methoxycarbonyl)caffeoyl]-meso-tartrate (3b) mp=72-74° C.; ¹H NMR (C): 7.59 (d, 16.0, 2H), 7.21-7.38 (m, 20H), 6.88 (s, 2H), 6.29 (d, 16.0, 2H), 6.02 (s, 2H), 3.91 (s, 6H), 3.90 (s, 6H); ¹³C(C): 164.9, 164.8, 153.0, 152.9, 144.6, 143.9, 142.7, 139.01, 138.98, 133.01, 128.6, 128.5, 128.27, 128.1, 127.3, 127.1(×2), 123.5, 122.6, 117.8, 79.6, 71.6, 55.9 (×2). Removal of the methoxycarbonyl groups by Procedure B gave bis(diphenylmethyl) dicaffeoyl-meso-tartrate (3c), mp=142-4° C. (dec.); ¹H NMR (M): 7.55 (d, 16.0, 2H), 6.82 (s, 2H), 6.77-7.25 (m, 20H), 6.20 (d, 16.0, 2H), 6.01 (s, 2H); 1C (M): 167.4d, 166.8d, 150.1d, 148.9u, 146.8d, 140.8 (×2)d, 129.6u, 129.5u, 129.2u, 129.1u, 128.2u, 128.1u, 127.3d, 123.5u, 116.5u, 115.4u, 113.4u, 80.8u, 72.8u. Removal of the diphenylmethyl groups by Procedure D gave meso-DCTA (3) whose ¹H and ¹³C NMR agreed with the literature except that C—I and C-2 of the tartaric acid portion were 3.5 upfield from that of an alleged meso-chicoric acid from Equisetum arvense. Since the latter was isolated by chromatography with 1% NH₃ in MeOH, it may have been the ammonium salt. Dissociation of a carboxyl group deshields both the carbonyl and α-carbon resonances by 3-4 ppm. This hypothesis was confirmed since the ¹³C-NMR of the ammonium salt of 3 matched that of the “meso chicoric acid” isolated from Equisetum which therefore was of the corresponding ammonium salt.

Dicaffeoyl-L-glyceric acid (19). Calcium L-glycerate dihydrate (Aldrich 37241-2) was dried to constant weight and reacted with 35a by Procedure E to give bis[di(methoxycarbonyl)caffeoyl]glyceric acid (19a), gum; ¹H NMR (C): 0.7.73 (d, 16.0, 1H), 7.66 (d, 16.0, 1H), 7.3-7.5 (m, 6H), 6.51 (d, 16.0, 1H), 6.44 (d, 16.0, 1H), 5.56 (m, 1H), 4.6-4.8 (m, 2H), 3.92 (s, 12H); ¹³CNMR (C): 171.9, 165.9, 165.4, 153.1, 153.0, 152.9 (×2), 144.6 (×2), 143.83 (×2), 143.79, 142.7, 133.2, 133.1, 126.90, 126.85, 123.5 (×2), 122.6, 122.5, 118.5, 117.9, 70.2, 62.9, 56.0 (×4). Hydrolysis of 19a by Procedure B but with a reaction time of only 10 min. gave 19, mp 234-236° C.; Anal. (C₂₁H₁₈O₁₀)C, H; ¹H NMR (D): 7.54 (d; 15.9, 1H), 7.51 (d, 15.9, 1H), 7.0-7.1 (m, 4H), 6.78 (dd, 1.6, 8.1, 2H), 6.37 (d, 15.9, 1H), 6.29 (d, 15.9, 1H), 5.36 (m, 1H), 4.56 (m, 2H); ¹³C (D): 168.5, 166.0, 165.6, 148.6, 148.5, 146.4, 146.0 (×2), 145.5, 125.2 (×2), 121.5, 121.4, 115.7 (×2), 115.0, 114.9, 113.0, 112.8, 70.2, 62.4.

Bis(3,4-dihydroxydihydrocinnamoyl)-L-tartaric acid (22). 3,4-Dihydroxydihydrocinnamic acid was reacted by Procedure A to give a mixture of 3,4-dimethoxycarbonyldihydrocinnamic acid (22a) and its anhydride which were separated by silica gel column chromatography and the latter hydrolyzed by 80% HOAC solution on a steam bath to give (22a): gum, ¹H NMR (C): 7.16 (d, 7.2, 1H), 7.14 (s, 1H), 7.10 (d, 7.2, 1H), 3.90 (s, 6H), 2.97 (t, 8.0, 2H), 2.69 (t, 8.1, 2H); ¹³C NMR (C): 177.5, 153.4 (×2), 142.2, 140.8, 139.4, 126.7, 123.0, 122.9, 55.8 (×2), 35.0, 29.9. Reaction of 22a by Procedure I gave the acid chloride which was subjected to Procedure G to give bis(3,4-dimethoxycarbonyldihydrocinnamoyl)-L-tartaric acid (22b): gum, ¹H NMR (C): 7.18 (d, 8.1, 2H), 7.10 (dd, 8.1, 1.9, 2H), 7.09 (d, 1.9, 2H), 5.55 (s, 2H), 3.92 (s, 6H), 3.91 (s, 6H), 2.98 (m, 4H), 2.81 (m, 4H); ¹³NMR (C): 171.0, 167.1, 154.7, 153.7, 142.0, 140.5, 139.3, 127.0, 123.2, 123.0, 70.3, 56.1, 56.0, 34.6, 30.1. Deprotection of 22b by Procedure B and preparative HPLC gave 22, as a gum which on analytical HPLC gave a single peak with a retention time of 2.5° (70% MeOH—H₂O+1% HOAc) or 3.1° (45% MeCN—H₂O+1% HOAc); ¹H NMR (D): 6.66 (bs, 2H), 6.54 (d, 8.0, 2H), 6.35 (bd, 7.5, 2H), 5.38 (s, 2H), 2.75 (m, 4H), 2.49 (m, 4H); ¹³C NMR (D): 172.2, 170.8, 145.5, 143.5, 131.4, 118.5, 115.6, 115.5, 74.8, 35.3, 29.5; HRFABMS: Calcd. m/z for C₂₂H₂₂O₂Na, 501.1069; Fd. 501.1006.

Digalloyl-L-tartaric acid (25). Gallic acid (3,4,5-trihydroxybenzoic acid) was reacted by Procedure A to give 3,4,5-trimethoxycarbonylbenzoic acid (25a) m.p.=143-144° C.; ¹HNMR (C): 7.99 (s, 2H), 3.94 (s, 6H), 3.93 (s, 3H); ¹³CNMR (C): 169.1, 152.6, 151.6, 143.8, 139.1, 127.8, 122.6, 56.4, 56.2. Procedure I converted 25a to the acid chloride which reacted with bis(diphenylmethyl) L-tartrate (1a) according to Procedure F to give bis(diphenylmethyl) bis(3,4,5-trimethoxycarbonylbenzoyl) L-tartrate (25b), gum: ¹H NMR (C): 7.62 (s, 4H), 7.30-7.36 (m, 20H), 6.96 (s, 2H), 6.08 (s, 2H), 3.94 (s, 6H), 3.92 (s, 12H); ¹³CNMR (C): 164.0, 162.5, 152.4, 151.5, 1143.6, 138.4, 138.1, 128.6, 128.54, 128.50, 128.3, 128.2, 127.5, 126.8, 126.5, 122.3, 79.3, 71.6, 56.4, 56.1. Sequential deprotection of 25b by Procedures D and B and preparative HPLC gave 25, as a gum which on analytical HPLC gave a single dominant peak with a retention time of 1.7′(10% MeOH-water+1% HOAc) or 4.8°, (10% MeCN—H₂O+1% HOAc); ¹H NMR (D): 7.01 (s, 4H), 5.70 (s, 2H); ¹³C NMR (D): 167.3, 164.8, 117.8, 108.8, 145.5, 139.1, 70.9; HRFABMS: Calcd. m/z for C₁₈H₁₄O₁₄Na, 477.0281; Fd. 477.0276.

Bis(3,4-dihydroxybenzoyl)-L-tartaric acid (26). 3,4-Dihydroxybenzoic acid (Aldrich 10,980-0) was reacted by Procedure A to give 3,4-dimethoxycarbonyl-benzoic acid (26a) as an off-white solid, m.p.=168-169° C.; ¹H NMR (C+M): 8.00 (dd, 8.6, 2.2, 1H), 7.99 (s, 1H), 7.39 (d, 8.6, 1H), 3.93 (s, 6H); ¹³C NMR (C+M): 167.0, 153.3, 153.0, 146.1, 142.3, 129.7, 128.8, 125.0, 123.1, 56.1 (×2). Procedure I converted 26a to the acid chloride which reacted by Procedure G to give bis(3,4-dimethoxycarbonylbenzoyl)-L-tartaric acid (26b), gum; ¹H NMR (C+M): 8.04 (dd, 8.4, 1.9, 2H), 8.07 (bs, 2H), 7.46 (d, 8.4, 2H), 6.00 (s, 2H), 5.78 (bs, 2H), 3.93 (s, 3H), 3.92 (s, 3H); ¹³CNMR (C+M): 167.5, 163.5, 152.7, 152.4, 146.3, 142.0, 128.5, 127.4, 124.6, 123.0, 71.8, 55.6 (×2). Deprotection of 26b by Procedure B and preparative HPLC gave 26 as a white solid, mp=175-176° C., Anal. (C₁₈H₁₄O_{12+1.5}H₂O)C, H; ¹H NMR (D): 9.97 (s, 2H), 9.56 (s, 2H), 7.41 (d, 2.5, 2H), 7.39 (d, 8.1, 2H), 6.87 (dd, 8.1, 2.5, 1H), 5.73 (d, 2.7, 2H); ¹³C NMR (D): 167.4, 164.5, 151.1, 145.1, 122.3, 119.1, 116.4, 115.4, 71.0.

Bis(3,4-dihydroxyphenylacetyl)-L-tartaric acid (28). 3,4-Dihydroxyphenyl-acetic acid was reacted by Procedure A to give 3,4-dimethoxycarbonylphenylacetic acid (28a); oil; ¹H NMR (C): 7.28-7.17 (m, 3H), 3.89 (s, 0.6H), 3.63 (s, 2H); ¹³C NMR (C): 175.7, 153.2 (×2), 142.1, 141.5, 132.5, 127.9, 124.0, 123.0, 55.8 (×2), 40.1. Reaction of 28a by Procedure H gave the acid chloride which was subjected to Procedure G to give bis(3,4-dimethoxycarbonylphenylacetyl)-L-tartaric acid (28b), gum; ¹H NMR (M): 7.35-7.20 (m, 3H), 5.72 (s, H-2), 3.85 (s, 6H), 3.80 (s, 2H);

¹³CNMR (M): 171.3, 168.9, 154.74, 154.69, 143.6, 143.0, 134.2, 129.1, 125.3, 1240.1, 72.7, 56.4 (×2), 40.3. Deprotection of 28b by Procedure B and preparative HPLC gave 28 as a gum; Anal. (C₂₀H₁₈O₁₂+H₂O), calcd, C, 51.3, H, 4.3; found, C, 51.5, H, 4.8; Analytical HPLC gave a single major peak with a retention time of 2.72° (10:1 MeCN:H₂O+1% HOAc) or 1.76° (5:1 MeOH:H₂O); ¹H NMR (M): 6.72-6.68 (m, 4H), 6.59(dd, 8.0, 1.9, 2H), 5.65 (s, 2H), 3.59 (s, 4H); ¹³C NMR (M): 172.6, 169.1, 146.2, 145.5, 126.2, 121.9, 117.6, 116.3, 72.4, 40.6.

Biochemical Characterization

Following synthesis and chemical characterization, all compounds were evaluated for capacity to inhibit IN activity in vitro, in the absence of cells, in the disintegration assay. All IC₅₀ values referred to and reported in Table 1 were determined with this assay.

Disintegration Assay.

The disintegration activities of IN in the presence and absence of inhibitors was assayed in vitro as modified from the method of Chow (Science 255:723-726 (1992)). The following oligonucleotides (GenoSys, Inc) were used as DNA substrates:

| | |
|---|---|
| T1 (16 mer): | 5′-CAGCAACGCAAGCTTG-3′; |
| T3 (30 mer): | 5′-GTCGACCTGCAGCCCAAGCTTGCGTTGCTG-3′; |
| V2 (21 mer): | 5′-ACTGCTAGAGATTTTCCACAT-3′; |
| V1/T2 (33 mer): | 5′-ATGTGGAAAATCTCTAGCAGGCTGCAGGTCGAC-3′. |

The oligonucleotides were gel purified by the manufacturer. Oligonucleotide T1 was labeled at the 5′-end using T4 polynucleotide kinase and [γ-³²P] ATP (3000 Ci/mmol, Amersham). The substrate for assaying disintegration activity, the Y-oligomer, was prepared by annealing the labeled T1 strand with oligonucleotides T3, V2, and V1/T2. In a 20 μl volume, the DNA substrate (0.1 pmol) was incubated with 1.5 pmol recombinant IN for 60 minutes at 37° C. in a buffer containing a final concentration of 20 mM HEPES pH 7.5, 10 mM DTT, 0.05% Nonidet P-40, and 10 mM MnCl₂. To each 19 μl of reaction mixture, 1 μl of inhibitor at various concentrations in solvent or solvent alone was added. The reaction was stopped by the addition of EDTA to a final 18 mM concentration. Reaction products were heated at 90° C. for 3 minutes before analysis by electrophoresis on a 15% polyacrylamide gel with 7M urea in Tris-borate-EDTA buffer. All reactions were performed at enzyme excess and reactions were stopped within the linear range of the reaction. Although it has been suggested by one group that the inhibitory effects of bis-catechols are metal ion dependent, all reactions were performed in the presence of MnCl₂ rather than MgCl₂. Our findings indicate that the DCTA's and DCQA's inhibit HIV IN whether Mg⁺⁺ or Mn⁺⁺ is the source of divalent cation and recent work from our groups suggests that metal ion is not required at all. These data are more consistent with the inhibitory activity of bis-catechols against avian sarcoma virus IN. All compounds were first tested at 25 μM. For active compounds, IC₅₀ analysis was determined from a median effect plot using CalcuSyn software (Biosoft, Cambridge, UK) on 0.5 log₁₀ dilutions of inhibitor in triplicate experiments.

Selectivity Against IN of Compounds 1, 22, 25, 26 and 28

As shown in Table 2, compounds 1, 22, 25, 26 and 28 demonstrated selectivity against IN when tested against two different molecular clones of HIV. The derivation of these clones and the manner in which this comparison was made is as follows.

Generation of L-Chicoric Acid Resistant Strain HIV_{NL4-3clone1-D4}

HIV_{NL4-3} plasmid (a gift from Dr. P. Krogstad, UCLA, Los Angeles, Calif.), was transfected in HeLa cells using Lipofectin® (Gibco/BRL). Excess DNA was removed by washing and cells were co-cultured with H9 cells for 18 hours. The H9 cells were removed and re-cultured in growth medium. When the culture was 100% positive for HIV antigens by indirect immunofluorescence, the virus was inoculated onto H9 cells and incubated at 37° C. for several weeks in the presence of 2 μM L-chicoric acid. When this culture was 100% positive the virus was isolated and one aliquot was passaged in a similar manner in 4 μM L-chicoric acid. Finally, virus was cultured in the presence of 8 μM L-chicoric acid and the resultant virus filter-clarified, aliquoted, and stored at −70° C.

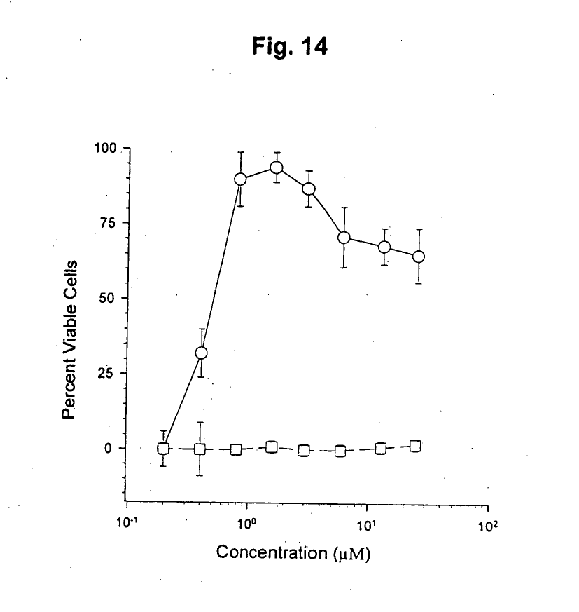

HIV_{NL4-3}, following culture in 8 μM L-chicoric acid, was tested for resistance to the anti-HIV activity of the compound using a cytopathicity-based first described by Montefiori et al. This assay takes advantage of the lytic nature of T-cell tropic clones of HIV and decreased cell viability in this assay has been shown to correlate well with HIV replication. The fifty-percent effective dose (ED₅₀) of L-chicoric acid against HIV_{NL4-3} control virus was 400 nM while HIV_{NL4-3} passaged in the presence of 8 μM L-chicoric acid was completely resistant to the compound (FIG. 14). Cloning and sequencing of virus from the HIV_{NL4-3} passaged in the presence of 8 μM L-chicoric acid was undertaken to determine the molecular basis for resistance to L-chicoric acid and to isolate a resistant HIV clone.

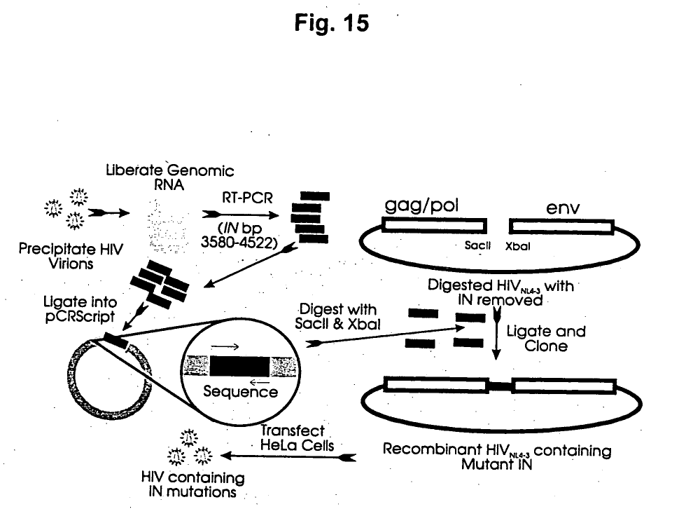

The overall cloning and sequencing strategy is illustrated in FIG. 15. For cloning and sequencing, HIV from 10 ml of culture was centrifuged at 33,000×g for 4 hours at 4° C. Virions were lysed and RNA isolated using Purescript® (Gentra, Frederick, Md.). Primers used to amplify cDNA under these conditions recognize the 5′ and 3′ ends of IN at nucleotide positions 3580-3605 (“INS” primer: 5′-ggtctccgcgggaatcaggaaagtac-3′) and 4497-4522 (“INX” primer: 5′-gcttttctagaaatatacatatggtg-3′) respectively, and generate a 943 base pair (bp) product. First strand synthesis using INX primer and Superscript II®, an avian myeloblastosis virus RT (Gibco/BRL), was performed at −42° C. for 50 minutes according to manufacturer's instructions. Thirty-eight cycle amplification was performed using Pfu thermostable DNA polymerase (Stratagene, La Jolla, Calif.) according to manufacturer's instructions. Optimum Mg⁺⁺ concentration for these studies was determined to be 1 mM. Conditions for polymerase chain reaction were: 96° C. for one minute, 40° C. for 30 seconds, 72° C. for 2 minutes for the first two cycles followed by 96° C. for one minute, 55° C. for one minute and 72° C. for 3 minutes for thirty-six cycles. The final cycle included a 10-minute, 70° C. elongation step. The resulting reverse transcriptase polymerase chain reaction (RT-PCR) products were separated by agarose gel electrophoresis and visualized by ethidium bromide staining. Appropriately sized products were eluted from the gel and blunt-end ligated into PCRScript® (Stratagene) for dideoxynucleotide sequencing using Sequenase II® (US Biochemical, Cleveland, Ohio) according to manufacturer's instructions. The entire integrase sequence was determined through the use of six oligonucleotide primers: INS, INX, Core 1,5′-cagctgtgataaatgtcagcta-3′ (nt3721-3741), Core 2: 5′-ccatttgtactgctgtcttaa-3′ (nt4122-4142), INSPF: 5′-gcaatttcaccagtactacagt-3′ (nt3962-3983), and INSPR: 5′-gtagggaatgccaaattcctg-3′ (nt4016-4036). Manual sequence analysis was confirmed by automated DNA sequencing.

Sequencing the integrase genes from both drug-resistant and control HIV_{NL4-3} demonstrated several mutations. Control virus contained two silent mutations at nucleotides 3832 and 4009. These silent mutations are believed to arise from a discrepancy in the published sequences of HIV_{NL4-3} and were likely not a result of passage of HIV in the absence of inhibitor. Drug-resistant HIV_{NL4-3} had the same silent mutations as well as a single G to A transition at nucleotide position 4025 leading to an amino acid change from glycine to serine at amino acid 140.

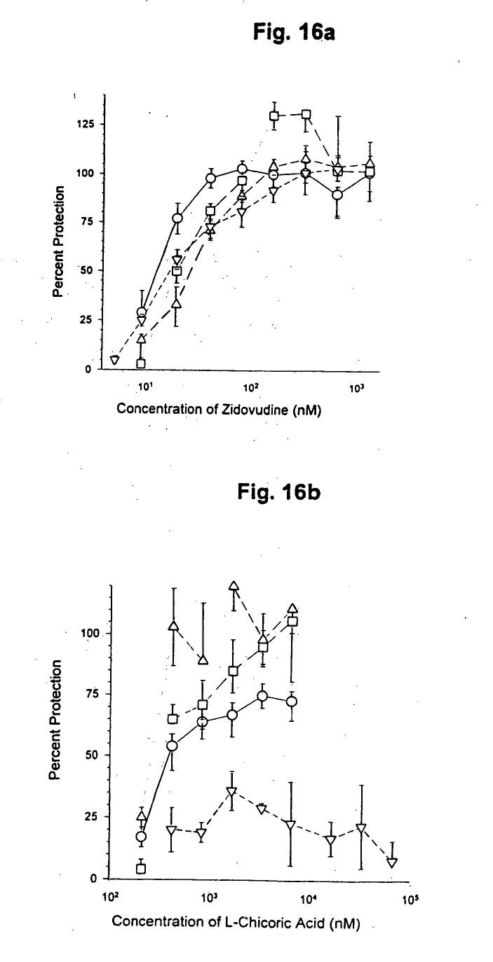

To determine whether this amino acid, change was responsible for the observed resistant phenotype, the integrase genes from drug-resistant HIV_{NL4-3} were cloned into the native HIV_{NL4-3} plasmid (pNL4-3). This cloning was accomplished through site-directed mutagenesis introducing several silent mutations immediately upstream and downstream of the integrase gene. These mutations generated two unique restriction sites: an upstream SacII and a downstream XbaI (FIG. 15). Introduction of these mutations allowed the entire integrase gene with only minimal upstream and downstream nucleotides to be digested and “swapped” between drug-resistant and drug-sensitive clones. Two clones, 7-1 and 7-3, containing control integrase genes, wildtype except for silent mutations generating the restriction sites, and Clone 1-D4, containing drug-selected integrase with the G140S mutation were chosen for further study. Once transfected into HeLa cells and amplified in H9 cells, the viruses from all three clones maintained the same sensitivity to zidovudine, a reverse transcriptase inhibitor, as the parental HIV_{NL4-3} (FIG. 16a). The three clones containing wild-type integrase (the control viruses, clones 7-1 and 7-3 and wild-type HIV_{NL4-3}) maintained the L-chicoric acid-sensitive phenotype. Clone 1-D4, on the other hand, was resistant to the anti-HIV effects of L-chicoric acid (FIG. 16b). The ED₅₀ for the drug-resistant clone was >600-fold higher than the drug-sensitive clones.

Amino acid 140 of integrase has not been mutated previously using site-directed mutagenesis. Furthermore, a search of the GenBank database does not indicate any naturally occurring mutations at this site. This amino acid is also highly conserved in integrases from other retroviruses, retrotransposons, and transposable elements of bacteria. Mutation at this site, from the highly conserved glycine to serine, has little effect on HIV replication but completely abrogates the anti-HIV activity of L-chicoric acid.

Assays, as described above, to determine the ED₅₀ for a given compound against the molecular clone HIV_{NL4-3clone1-D4} and the parental clone HIV_{NL4-3} give an indication of the selectivity of the compound for inhibition of HIV replication through IN. Resistance to a compound, as measured by an increased ED₅₀, by clone HIV_{NL4-3clone1-D4}, implies selectivity of the compound for IN. Thus clone HIV_{NL4-3clone1-D4} can be used to screen candidate IN inhibitors for selectivity.

Assessing Capacity of L-CCA to Act Synergistically

Dual Antiviral Agent Measurements.

Molecular clones of HIV included wild-type HIV_{NL4-3}, HIV_{NL4-3 M184V}, and HIV_{NL4-3} JF26/A7, All three clones were a generous gift from P. Krogstad (UCLA, Los Angeles, Calif.). Viruses were initially, transfected in adherent HeLa cells using Lipofectin®. After 48 hours, H9 cells were added. Following 24 hours of co-culture, non-adherent cells were removed and cultured. Cells were monitored by indirect immunofluorescence and RT release as described below until the culture was 100% infected by HIV-1. Supernatant fluids were collected and clarified of cells by low-speed centrifugation followed by filtration through 0.45 μm filters.

RT Assay and Immunofluorescence Analysis.

Each culture supernatant was precipitated with 0.42 ml of 30% polyethylene glycol as described previously by Robinson et al. (J. Acquired Immune Defic. Syndr. 2: 33-42 (1989)). Precipitated virus was lysed and incorporation of [³H]-thymidine into poly rA-oligo dT templates was measured according to a modification by Robinson (ibid.) of the method first described by Poiesz et al (Proc. Natl. Acad. Sci. USA. 77: 7415-7419 (1980)). Trichloroacetic acid precipitable raw cpm were determined on a Beckman O-scintillation counter. The mean cpm for the triplicate infections was determined and mean background cpm from three cell control cultures run in parallel to each assay were subtracted. The resultant corrected cpm were multiplied by 8 to convert to cpm/ml of culture supernatant fluid. For immunofluorescence analysis, cells from triplicate wells were combined and spotted onto glass slides. The percentage of cells expressing HIV antigens was quantitated using pooled human anti-HIV serum followed by fluorescein-conjugated goat anti-human IgG and observation under fluorescence microscopy as described by Robinson (ibid.).

Synergy Analysis

Compounds:

Zidovudine, DDC, and 2′,3′-dideoxyinosine (DDI) were purchased from Sigma Chemical Co (St. Louis, Mo.). All three were reconstituted to 1 mM stock solutions in deionized water and stored at −20° C. until use. Michael Melnick (Agouron Pharmaceuticals, San Diego) provided Nelfinavir. Manfred Reinecke (Texas Christian University, Fort Fort Worth, Tex.) provided L-chicoric acid. Both were reconstituted in deionized water and stored at −70° C. until use. All stocks were diluted in growth medium and filter-clarified before being tested for cell toxicity and anti-HIV activity.

Anti-HIV Assays:

Anti-HIV activity of compounds was determined both alone and in combination using a cytopathicity based assay as described above. This assay utilizes Finter's neutral red dye; protection from HIV-induced cell death is highly correlated with HIV-1 antigen synthesis, RT release, and the formation of infectious progeny virions. All drugs were tested at concentrations well below their toxic doses either alone or in combination. The fifty-percent effective dose (ED₅₀) was calculated for triplicate infections. Mean ED₅₀ (shown in FIG. 5) were calculated for each drug against each HIV variant from a minimum of three experiments performed in triplicate.

Mixed Dose Effect Analyses:

Mixed dose effect analyses were performed using the method of Chou and Talalay (Adv. Enzyme Regul. 22: 27-55 (1984)) and commercially available software: CalcuSyn for Windows (Biosoft, Ferguson, Mo., USA). Experiments were designed at a fixed ratio of drugs; the ratio for each drug combination was determined based on the ED₅₀ of each drug alone against each HIV variant. Combination indices were calculated on representative experiments performed in triplicate according to the following formula of Chou and Talalay:
fa/fu=(D/Dₘ)^{m}

Where fa is the fraction affected by the dose, fu is the unaffected fraction, D is the dose of the drug, Dₘ is the median-effect dose determined to be the x-intercept of the median-effect plot, and m is the sigmoidicity of the dose-effect curve, determined by the slope of the median-effect plot. The median effect plot is based on the following equation:
log(fa/fu)=^{m log} (D)−m log (Dₘ)

This equation yields a straight-line y=mx+b; therefore, the x intercept is the log (Dₘ) value and the slope is m.

The combination index (CI) is based on the multiple drug-effect equation of Chou and Talalay where a CI=1 is additive, greater than 1 is antagonism and less than 1 is synergism. This equation, first defined by Chou and Talalay (Trends Pharmacol. Sci. 4: 450-454 (1983)) is:

$$CI = \frac{{( D )}_{1}}{{( {D}_{x} )}_{1}} + \frac{{( D )}_{2}}{{( {D}_{x} )}_{2}}$$

Where D₁ is the dose of drug 1 and D₂ is the dose of drug 2 in combination that results in x % inhibition and (Dₓ)₁ and (Dₓ)₂ are the doses of drug 1 and drug 2 alone that result in x % inhibition. The results of mixed drug analyses for the pairwise combinations of L-CCA plus DDC, L-CCA plus nelfinavir, and L-CCA plus zidovudine are shown in Tables 4-6 respectively.

Triple Antiviral Agent Measurements:

Anti-HIV Compounds:

ZDV was purchased from Sigma Chemical Company (St. Louis, Mo.) and was reconstituted with cold deionized water to a final concentration of 1 mM. Protease inhibitor (PI) (AG1350) (Agouron Pharmaceuticals, La Jolla, Calif.) and reconstituted in 14% ethanol/37.5% DMSO/48.5% H₂O to a final concentration of 7 mM. AG1350 is slightly (less than 10-fold) less active than the recently FDA-approved PI, Viracept®. The L-CCA was dissolved in H₂O to a final concentration of 2.1 mM.

Cells and Virus.

The two clinical isolates were obtained from patients visiting the UCI Medical Center AIDS Clinic under an IRB-approved protocol. HIV_{R19} and HIV_{R45} were obtained approximately three weeks after inoculation with 100 μl of freshly drawn serum onto MT-2 cells (the time required for the MT-2 cells to become infected). Previous work has indicated that such isolates of HIV are predominantly syncytium-inducing, rapid-growing, T-cell-tropic isolates of HIV. Cell-free supernatants of HIV_{R19} and HIV_{R45} from MT-2 cells were inoculated onto phytohemagglutinin-stimulated PMBCs, cultured in growth medium supplemented with 11.5% FBS and 20 units of recombinant human IL-2/ml (Boehringer-Mannheim, Indianapolis, Ind.), and HIV was isolated at peak RT production (7 and 10 days). Both isolates were highly cytopathic and grew to similar titers with similar replication kinetics in tissue culture. The resulting limited passage, cell-free supernatant was directly inoculated onto MT-2 cells for use in antiviral assays.

Cell Toxicity and Anti-HIV Assays:

Cell toxicity and anti-HIV assays were performed as reported above. Briefly, compounds were diluted 1:1 in growth medium, filter sterilized, and further two-fold serially diluted from 1:8 to 1:1280 in triplicate wells of a microtiter plate. To each 50 μl of diluted drug, 50 μl of growth medium was added followed by 00 μl of MT-2 cell suspension (2×10⁵ cells). Cells were incubated with drug for 48 hours at 37° C., then harvested for cell viability in a neutral red dye assay as described. Similar toxicities were also seen if the cells were incubated for 72 hours prior to harvest.

Anti-HIV assays were performed as described above. Based upon cell toxicity data, compounds were diluted in growth medium such that a final 1:4 dilution of the sample would result in a concentration of sample that inhibited MT-2 cell growth by 5% (5% lethal dose, LD₅). The compounds were then two-fold serially diluted in triplicate. To each 50 μl of diluted compound, 50 μl of HIV_{LAI} was added and the virus-drug mixture was incubated for 1 hr at 37° C. Next, 100 ll of MT-2 cell suspension (2×10⁵ cells) was added to each well and cells were incubated for 72 hr at 37° C. Final multiplicity of infection (MOI) was 1-5. Cells were harvested to quantitate cytopathic effect using a neutral red dye assay as described. The antiviral concentration reported is the concentration of drug necessary to protect MT-2 cells from fifty percent viral-induced cell death; this is referred to as the fifty percent effective dose (ED₅₀).

The results reported herein clearly demonstrate that the addition of a first generation IN inhibitor to regimens using either a PI or ZDV improves upon the in vitro anti-HIV effect of that regimen. For all of the triple combination studies (FIGS. 6-9), all three inhibitors were used at suboptimal concentrations (below the ED₅₀). In addition, if L-CCA is added to ZDV and a PI, one can effectively reduce by at least 33% the amount of ZDV and PI required to provide an equivalent anti-HIV effect. Iii vitro combination analyses may or may not predict how combinations of antiviral agents will behave in vivo. For HIV, combinations of protease inhibitors and reverse transcriptase inhibitors demonstrated additive to synergistic effects in vitro, which were similar to their in vivo effects. With one major limiting factor in anti-HIV combination therapy being the cost of the antiviral agents, especially PIs, such a reduction could substantially increase the number of patients who could be on combination therapy regimens. The results of these in vitro studies, if they were to translate into in vivo results, could have a substantial impact on both the efficacy and costs of newer anti-HIV therapies.

The addition of L-chicoric acid to either zidovudine or protease inhibitor improved upon the observed anti-HIV activity of either compound alone. When all three drugs were combined, the anti-HIV activity was substantially better than either of the three compounds alone or any combination of two inhibitors. Doses of both zidovudine and protease inhibitor could be reduced by more than 33% for an equivalent anti-HIV effect if L-chicoric acid was added. The improved anti-HIV activity was observed with a tissue culture adapted strain of HIV (HIV_{LAI}) and with limited passage clinical isolates of HIV (HIV_{R19} and HIVR₄₅). These data demonstrate that a first generation HIV integrase inhibitor is at least additive and probably synergistic in combination with existing multi-drug regimens. The modified integrase inhibitors disclosed herein show similar synergistic properties. It is expected that the variations in molecular structure will prove advantageous in actual patient trials. For example, replacing ester linkages with more stable amide or even aliphatic linkages significantly improves the effectiveness of modified integrase inhibitors. A version of compound 36 in which the amide bonds are replaced with ester bonds is virtually ineffective Many alterations and modifications may be made by those having ordinary skill in the art without departing from the spirit and scope of the invention. Therefore, it must be understood that the illustrated embodiment has been set forth only for the purposes of example and that it should not be taken as limiting the invention which is properly defined by the following claims. The words used in this specification to describe the invention and its various embodiments are to be understood not only in the sense of their commonly defined meanings, but to include by special definition in this specification structure, material or acts beyond the scope of the commonly defined meanings. Thus if an element can be understood in the context of this specification as including more than one meaning, then its use in a claim must be understood as being generic to all possible meanings supported by the specification and by the word itself.

1. A method for treating HIV infections comprising administering a mixture of a reverse transcriptase inhibitor, and/or a protease inhibitor and an integrase inhibitor.

2. The method of claim 1 further comprising administering more than one reverse transcriptase inhibitor and/or more than one protease inhibitor.

3. The method of claim 1 further comprising administering more than one integrase inhibitor.

4. The method of claim 2 further comprising administering more than one integrase inhibitor.

5. The method of claim 1, wherein the integrase inhibitor is selected from a group consisting of chicoric acid 2,3-di(3,4-dihydroxy-dihydroxydihydrocinnamoyl)-tartaric acid, 2,3-di-(3,4-dihydroxybenzoyl)-tartaric acid, 2,3-di-(3,4-dihydroxyphenylacetyl)-tartaric acid, 2,3-di-(3,4,5-trihydroxybenzoyl-tartaric acid, 2,3-dicaffeoyldiamidopropionic acid, 1,2,-dicaffeoyl-glyceric acid, bis,-3,4-dicaffeoyldiamidobenzoic acid, di-3,4-dihydroxybenzylidene succinic acid, di-3,4-dihydrodihydroxybenzylidine succinic acid, 2,3-dicaffeoyl-serine, bis-dicaffeoyl-isoserine and 1,4-dicaffeoyl-lysine

6. The method of claim 1, wherein the reverse transcriptase inhibitor is selected from a group consisting of 2′,3′-dideoxycytidine, 2′,3′-dideoxyinosine and zidovudine.

7. The method of claim 1, wherein the protease inhibitor is Nelfinavir.

8. A composition for treating HIV infections comprising a mixture of a reverse transcriptase inhibitor, and/or a protease inhibitor and an integrase inhibitor.

9. The composition of claim 8, wherein the integrase inhibitor is selected from a group consisting of chicoric acid 2,3-di(3,4-dihydroxy-dihydroxydihydrocinnamoyl)-tartaric acid, 2,3-di-(3,4-dihydroxybenzoyl)-tartaric acid, 2,3-di-(3,4-dihydroxyphenylacetyl)-tartaric acid, 2,3-di-(3,4,5-trihydroxybenzoyl-tartaric acid, 2,3-dicaffeoyldiamidopropionic acid, 1,2,-dicaffeoyl-glyceric acid, bis,-3,4-dicaffeoyldiamidobenzoic acid, di-3,4-dihydroxybenzylidene succinic acid, di-3,4-dihydrodihydroxybenzylidine succinic acid, 2,3-dicaffeoyl-serine, bis-dicaffeoyl-isoserine and 1,4-dicaffeoyl-lysine

10. The composition of claim 8, wherein the reverse transcriptase inhibitor is selected from a group consisting of 2′,3′-dideoxycytidine, 2′,3′-dideoxyinosine and zidovudine.

11. The composition of claim 8, wherein the protease inhibitor is Nelfinavir.

12. The composition of claim 8 further comprising more than one reverse transcriptase inhibitor and/or more than one protease inhibitor.

13. The composition of claim 8 further comprising more than one integrase inhibitor.

14. The composition of claim 12 further comprising more than one integrase inhibitor.

15. An integrase inhibitor selected from a group consisting of 2,3-di(3,4-dihydroxy-dihydroxydihydrocinnamoyl)-tartaric acid, 2,3-di-(3,4-dihydroxybenzoyl)-tartaric acid, 2,3-di-(3,4-dihydroxyphenylacetyl)-tartaric acid, 2,3-di-(3,4,5-trihydroxybenzoyl-tartaric acid, 2,3-dicaffeoyldiamidopropionic acid, 1,2,-dicaffeoyl-glyceric acid, bis,-3,4-dicaffeoyldiamidobenzoic acid, di-3,4-dihydroxybenzylidene succinic acid, di-3,4-dihydrodihydroxybenzylidine succinic acid, 2,3-dicaffeoyl-serine, bis-dicaffeoyl-isoserine and 1,4-dicaffeoyl-lysine

16. An integrase inhibitor having the formula: 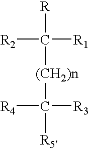
wherein n is between 0 and 4;
wherein R₁ and R₃ are selected from the group consisting of hydrogen, OR₆, NR₆ and aralkyl groups;
wherein R₇ is selected from the group consisting or hydrogen, alkyl and aralkyl;
wherein R and R₅ are selected from the group consisting of hydrogen, COOR₇ and CONHR₇;
wherein R₆ is 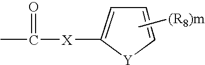
wherein X is a hydrocarbyl group with from 0 to 10 carbon atoms, Y is selected from CH═CH, n=CH, CH═N, O, S, or NR₇. m is between 0 and 3, and R₈ is selected from the group consisting of hydrogen, hydroxy, halo, lower alkoxy, alkycarbonyloxy and alkoxycarbonyloxy or a cyclic carbonate group with hydroxy groups on adjacent carbons; and
wherein R₂ and R₄ are hydrogen.

17. The integrase inhibitor of claim 16, wherein R₂ and R₄ combine with each other to form a cycloalkyl ring.

18. The integrase inhibitor of claim 16, wherein R₂ and R₄ are combined with R₁ and R₄, respectively, to form aromatic rings.

19. The integrase inhibitor of claim 18, wherein the aromatic rings are substituted with from one to three substituents selected from OR₆ and NR₆ groups.

20. The integrase inhibitor of claim 16, wherein when R and R₅ are COOR₇ or CONHR₇, and R₁, R₂ and R₃, R₄ combine to form an arylidene group.

21. The integrase inhibitor of claim 20, wherein the arylidene group is substituted with from 1 to 3 substituents selected from the, group consisting of hydroxy, halo, alkoxy, alkycarbonyloxy and alkoxycarbonyloxy.

22. An integrase inhibitor having the formula: 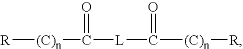
wherein R is 1,4-dicaffeoyl, n is between 0 and 6 and L comprises an amino acid linked by an ester or amide bond.

23. An integrase inhibitor having the formula: 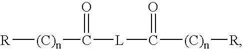
wherein R is 1,4-dicaffeoyl, n is between 0 and 6 and L comprises a chain of between 1 and 6 carbon atoms.
